(19) United States
(12) Patent Application Publication
(10) Pub. No.: US 2022/0072327 A1
(43) Pub. Date: Mar. 10, 2022

(54) SYMPHONIC PULSED ELECTROMAGNETIC FIELD THERAPY

(71) Applicant: HAELOX, LLC, McLean, VA (US)

(72) Inventors: Jeff Monroe, Mclean, VA (US); Wilson E. Taylor, Austin, TX (US); Larry Eugene Hand, Meridian, MS (US); Kimberly McGeorge, King, NC (US); Icasiana Barrs, Encino, CA (US); Joanne Wilkie, Encino, CA (US); Byanka McClain, Encino, CA (US); Srinivasa Boppana, Encino, CA (US)

(73) Assignee: HAELOX, LLC, McLean, VA (US)

(21) Appl. No.: 17/447,234

(22) Filed: Sep. 9, 2021

Related U.S. Application Data
(60) Provisional application No. 63/261,025, filed on Sep. 9, 2021.
(60) Provisional application No. 63/075,864, filed on Sep. 9, 2020.

(51) Int. Cl. A61N 2/00; A61N 2/02; A61M 21/02

(52) U.S. Cl. CPC A61N 2/002; A61N 2/008; A61M 2021/0027; A61M 21/02; A61N 2/02

(57) ABSTRACT

Apparatus and associated methods relate to a symphonic pulsed electromagnetic field (PEMF) therapy device operably coupled to a handheld solenoidal coil. In an illustrative example, the symphonic PEMF therapy device may be configured to drive the handheld solenoidal coil to generate a time-varying symphonic PEMF. The symphonic PEMF may, for example, be generated in response to activation of a predetermined symphonic frequency profile (PSFP) selected from a library of PSFPs. Each of the PSFPs may include a frequency set of, for example, at least 50 frequencies. The frequencies may, for example, include frequencies from at least four decades starting at 10 Hz. The PEMF therapy device may advantageously generate symphonic PEMF in response to user input selection, for example, of a predetermined medical therapeutic and/or a non-medical optimization effect.

### CROSS-REFERENCE TO RELATED APPLICATIONS

This application claims the benefit of U.S. Provisional Application Ser. No. 63/075,864, titled “SYMPHONIC PULSED ELECTROMAGNETIC FIELD THERAPY,” filed by Monroe, et al., on Sep. 9, 2020.

This application claims the benefit of U.S. Provisional Application Ser. No. 63/261,025, titled “Symphonic Pulsed Electromagnetic Field Therapy,” filed by Monroe, et al., on Sep. 9, 2021.

This application incorporates the entire contents of the foregoing application(s) herein by reference.

### TECHNICAL FIELD

Various embodiments relate generally to systems and methods configured to generate therapeutic electromagnetic field therapy according to predetermined compound frequency sets.

### BACKGROUND

People and animals who suffer from pain or injury have been provided with various traditional therapies and intervention including medication, physical therapy, and surgery. Similarly, immune system optimization has been supported by supplements, vitamins, and various therapies.

Pulsed electromagnetic field (PEMF) therapy, or low magnetic field stimulation, has been used to employ electromagnetic fields during treatment of non-fusing bone fractures in human and veterinary medicine, as well as various other forms of therapy and wellness promotion.

### SUMMARY

Apparatus and associated methods relate to a symphonic pulsed electromagnetic field (PEMF) therapy device operably coupled to a handheld solenoidal coil. In an illustrative example, the symphonic PEMF therapy device may be configured to drive the handheld solenoidal coil to generate a time-varying symphonic PEMF. The symphonic PEMF may, for example, be generated in response to activation of a predetermined symphonic frequency profile (PSFP) selected from a library of PSFPs. Each of the PSFPs may include a frequency set of, for example, at least 50 frequencies. The frequencies may, for example, include frequencies from at least four decades starting at 10 Hz. The PEMF therapy device may advantageously generate symphonic PEMF in response to user input selection, for example, of a predetermined medical therapeutic and/or a non-medical optimization effect.

Various embodiments may achieve one or more advantages. For example, some embodiments may be directed to systems and methods that advantageously select, transmit, and implement PSFPs designed for various beneficial therapeutic purposes. Some embodiments may be directed to generating interfaces that advantageously receive input from a user to discover, select, retrieve, and transmit a desired PSFP. Some embodiments may be directed to PEMF therapy devices that advantageously receive and implement PSFPs to generate SPEMFs defined thereby. Some embodiments may advantageously implement non-medical PSFPs to improve physical and mental performance and optimization. Some embodiments may advantageously implement PSFPs to apply medical therapy.

The details of various embodiments are set forth in the accompanying drawings and the description below. Other features and advantages will be apparent from the description and drawings, and from the claims.

### BRIEF DESCRIPTION OF THE DRAWINGS

FIG. 1A depicts an exemplary symphonic frequency therapy device (SFTD) in an illustrative use-case.

FIG. 1B depicts an exemplary symphonic frequency therapy system.

FIG. 1C depicts an exemplary method of selecting PSFP(s) for generating a symphonic PEMF.

FIG. 2A depicts an exemplary interface for selecting symphonic frequency profiles.

FIG. 2B depicts an exemplary interface for initiating generation of a selected symphonic frequency profile.

FIG. 2C depicts an exemplary non-medical interface for selecting non-medical symphonic frequency profiles.

FIG. 2D depicts an exemplary non-medical interface for initiating generation of a selected non-medical symphonic frequency profile.

FIG. 3 depicts an exemplary symphonic frequency therapy device.

FIG. 4A depicts a first exploded view of an exemplary symphonic frequency therapy device.

FIG. 4B depicts a second exploded view of the exemplary symphonic frequency therapy device of FIG. 4A.

FIG. 5A depicts a top plan view of an exemplary solenoidal coil which transduces electric signals from a SFTD into a toroidal shaped electromagnetic field of symphonic pulsed sine waves according to a predetermined symphonic frequency profile.

FIG. 5B depicts a side elevation view of the exemplary solenoidal coil of FIG. 5A.

FIG. 5C depicts a cross-section A view of the exemplary solenoidal coil of FIG. 5A.

FIG. 6 depicts a block diagram of an exemplary symphonic frequency therapy device.

FIG. 7 depicts exemplary predetermined symphonic frequency profiles.

FIG. 8 depicts an exemplary implementation of a symphonic frequency therapy device.

FIG. 9A depicts a top view of an exemplary implementation of a solenoidal coil.

FIG. 9B depicts an end view of the exemplary solenoidal coil of FIG. 9A.

FIG. 9C depicts a schematic of the exemplary solenoidal coil of FIG. 9A.

FIG. 10A, FIG. 10B, FIG. 10C, FIG. 10D, FIG. 10E, FIG. 10F, and FIG. 10G depict a perspective view, a top view, a rear view, a left-side view, a front view, a right-side view, and a bottom view of an exemplary PEMF therapy system.

FIG. 11A, FIG. 11B, FIG. 11C, FIG. 11D, FIG. 11E, FIG. 11F, and FIG. 11G depict a perspective view, a top view, a rear view, a left-side view, a front view, a right-side view, and a bottom view of an exemplary symphonic frequency therapy device.

FIG. 12A, FIG. 12B, FIG. 12C, FIG. 12D, FIG. 12E, FIG. 12F, and FIG. 12G depict a perspective view, a top view, a rear view, a left-side view, a front view, a right-side view, and a bottom view of an exemplary handheld solenoidal coil.

Like reference symbols in the various drawings indicate like elements.

### DETAILED DESCRIPTION OF ILLUSTRATIVE EMBODIMENTS

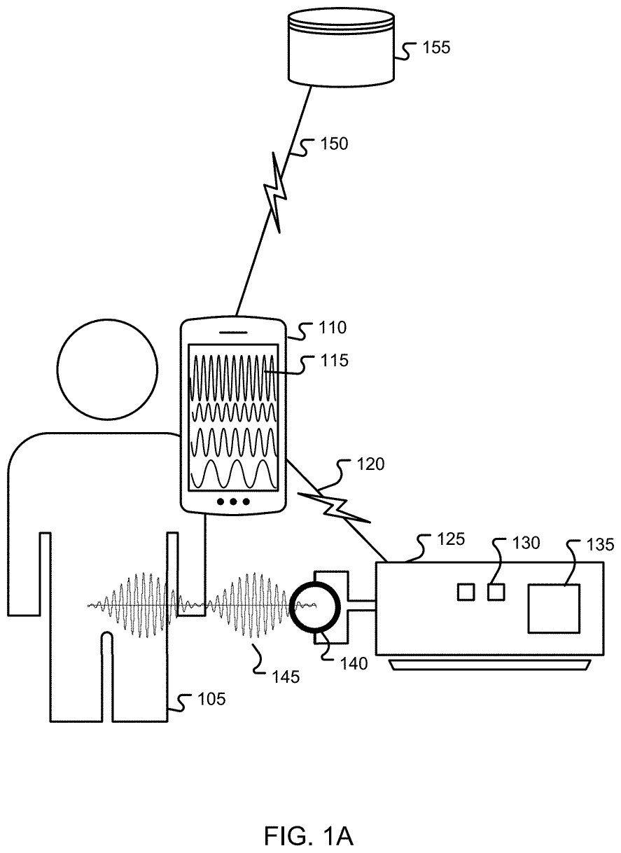

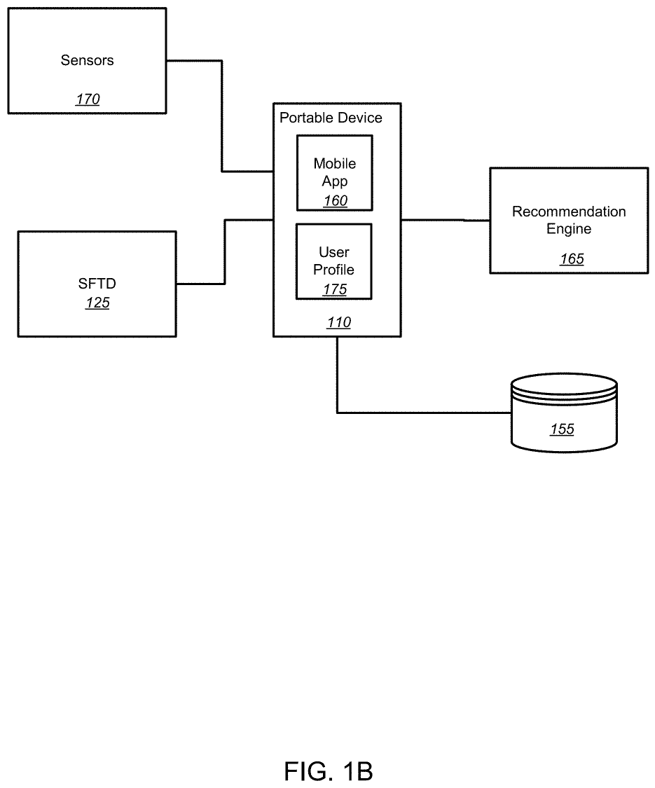

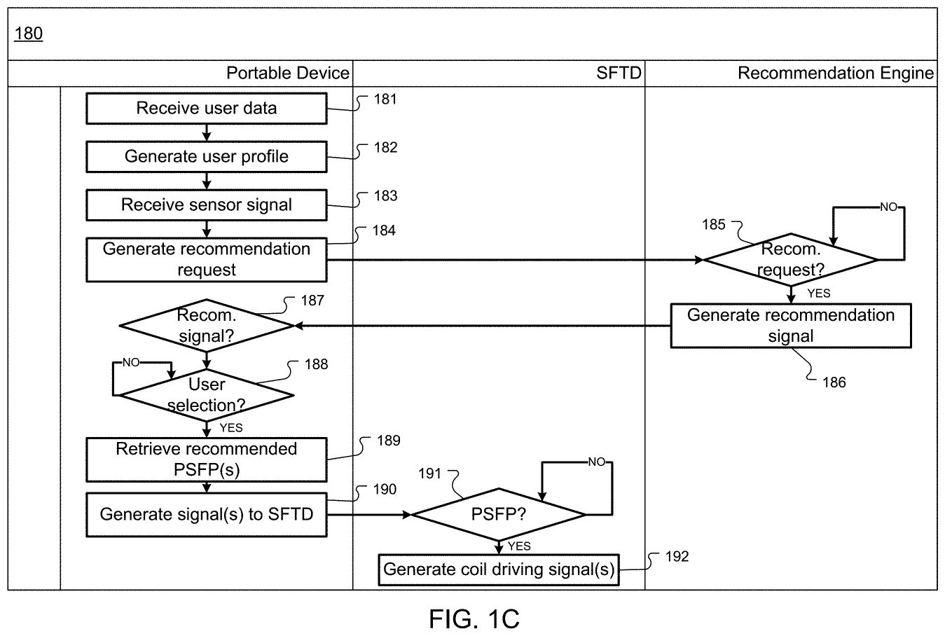

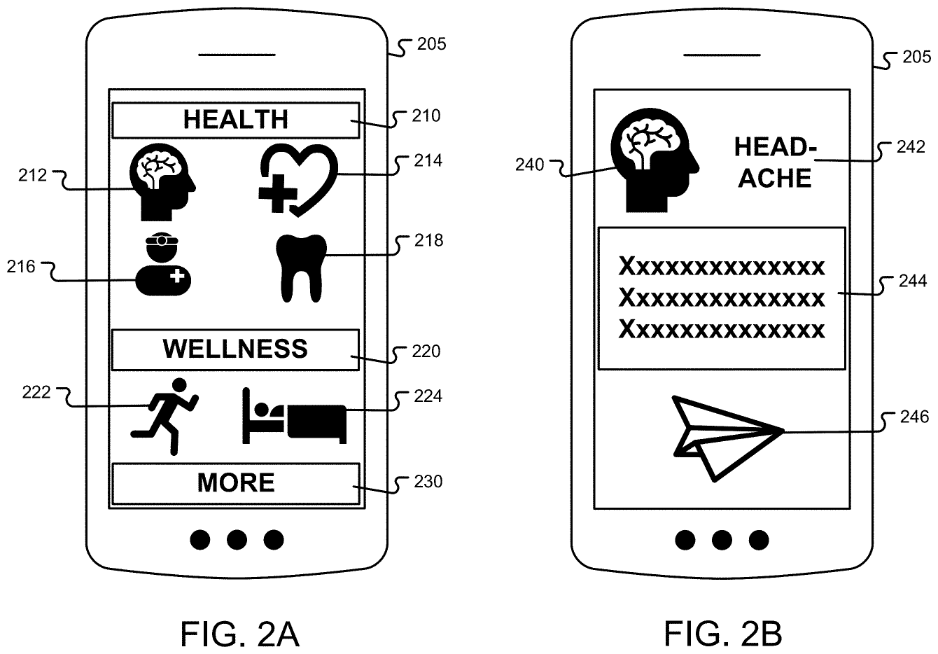

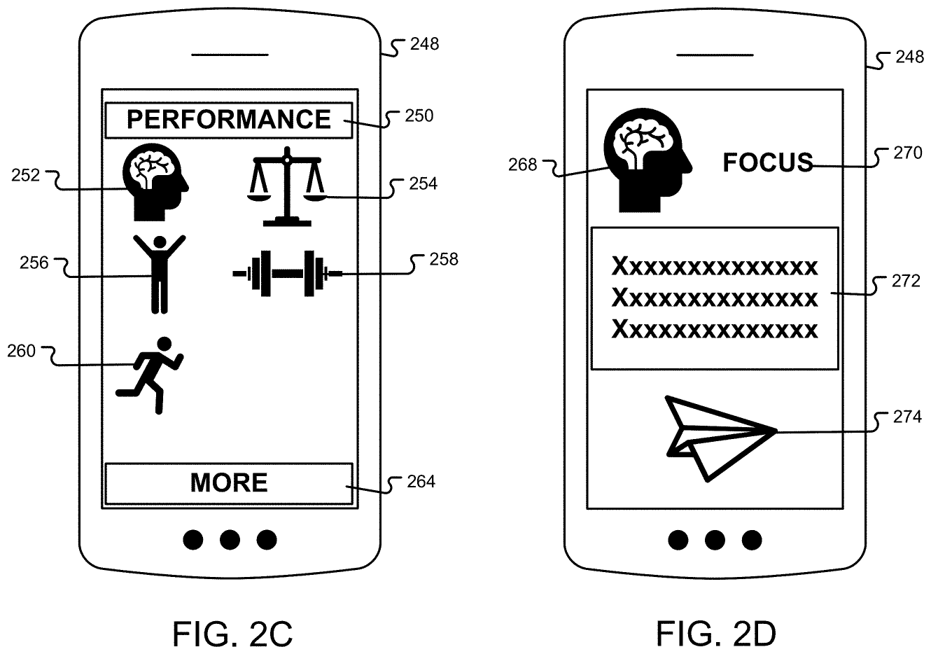

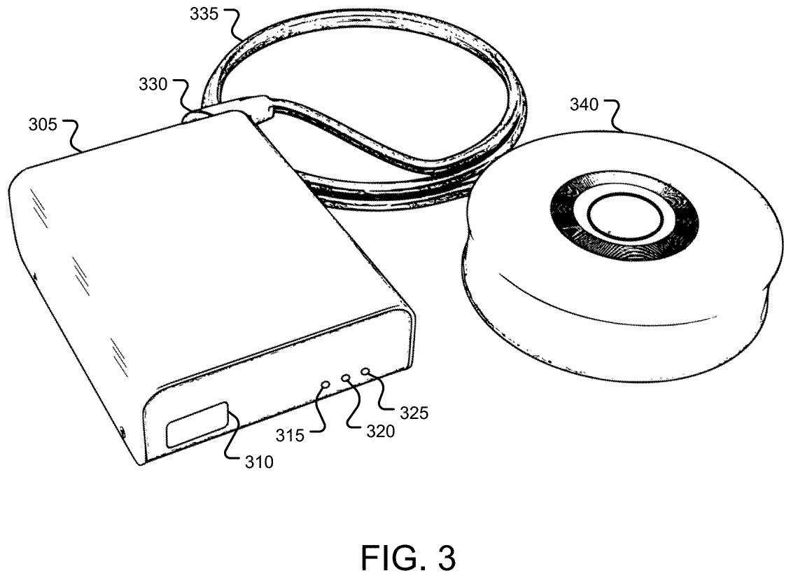

To aid understanding, this document is organized as follows. First, an illustrative use case scenario for selecting and generating predetermined symphonic frequency profiles is briefly introduced with reference to FIG. 1A. Second, an exemplary symphonic frequency therapy system is described with reference to FIG. 1B. Next, with reference to FIGS. 2A-2B, the discussion turns to exemplary embodiments that illustrate an implementation of laying out interfaces for selecting and initiating generation of predetermined symphonic frequency profiles. Finally, with reference to FIGS. 3-6E, the discussion turns to exemplary embodiments that illustrate implementations of symphonic frequency therapy devices.

FIG. 1A depicts an exemplary symphonic frequency therapy device in an illustrative use-case. A user 105 may browse or search predetermined symphonic frequency profiles (PSFPs) via interface 115 on a portable device 110. For example, the portable device 110 may be configured to generate and transmit a request for PSFPs. In the depicted example, as an illustration without limitation, a library of PSFPs (not shown) is stored in a remote data store 155. For example, the portable device 110 may transmit a request via communication link 150 to the remote data store 155, and retrieve at least one PSFP from the remote data store 155. In this example, the portable device 110 is further configured to send a command, via communication link 120, to initiate generation of the selected PSFP by a symphonic frequency therapy device (SFTD) 125. In some implementations, the communication link 120 may be established with a short-range wireless technology. For example, the short-range wireless technology may use the Bluetooth communication standard for communication and exchanges.

In this example, the SFTD 125 transmits an electrical signal of which handheld solenoidal coil 140 transduces into a symphonic pulsed electromagnetic field (SPEMF 145) composed of N superimposed sine waves at predetermined fixed frequencies f₁, f₂, . . . f_{N}. The SFTD 125 may indicate a current state via indicators 130, which may include, for example, individual LEDs or other lights and/or a dynamic display screen 135. In some examples, the handheld solenoidal coil 140 may be substantially of a weight of 5.5 lbs or less. For example, a weight of less than or equal to 5.5 pounds may advantageously promote portability.

In some examples, a SPEMF may advantageously affect a subject, especially compared, for example, to a singular frequency PEMF. PSFP parameters may include, by way of example and not limitation, a number of unique sine waves to generate. The PSFP may, by way of example and not limitation, define for each unique sine wave the frequency, phasing, amplitude, coefficient, or some combination thereof of each sine wave. In some implementations, the number of superimposed sine waves, N, may be at least 4, for example, to produce energy at predetermined sets of frequencies to achieve a target therapeutic response, for example, by the user. In various embodiments, for example, the PSFP selected by a user may have N be 3, 4, 5, 6, 7, 8, 9, or up to at least about 50 distinct frequencies at a time. In various embodiments, the frequency set defined by the PSFP may be within a range of at least 10¹ Hz-10⁴ Hz. In various embodiments, the PSFP may include a continuous stream characterized by the summation, Σᵢ₌₁^{N}aᵢ*fᵢ, of N sinusoidal components, each component i at a corresponding fixed predetermined amplitude corresponding to a predetermined coefficient aᵢ. The predetermined amplitude, for example, may be defined in a range of at least four orders of magnitude. The coefficient aᵢ may, for example, define amplitude and/or phase corresponding to the specific frequency fᵢ. In various embodiments the frequencies may include at least one frequency from each of multiple decades. For example, in some embodiments the frequencies may include at least one frequency from each of four decades starting at 10 Hz. For example, the frequencies may include K₁*10¹ Hz, K₂*10² Hz, K₃*10³ Hz, and K₄*10⁴ Hz, where Kᵢ<10. As an illustrative example, the frequencies may include, by way of example and not limitation, 20 Hz (K₁=2), 133 Hz (K₂=1.33), 8560 Hz (K₃=8.560), and 10436 Hz (K₄=1.0436).

Some implementations may include two or more distinct PSFPs that share identical sets of frequencies fᵢ, but the relative amplitudes of those frequencies may be substantially different among the distinct PSFPs to produce a different desired therapeutic effect. Some embodiments may have an initial predetermined fixed phase relationship between or among one or more of the frequencies fᵢ. Accordingly, a user may advantageously select a PSFP specific to a current therapeutic desire and/or need and initiate a SPEMF therapy composed of a carefully selected symphony of pure sine waves sent through the coil 140 of the SFTD 125 according to parameters provided in the PSFP.

The portable device 110 may be previously provided with one or more PSFPs for a user to select, such as, for example, default PSFPs, popular PSFPs, previously selected PSFPs, purchased PSFPs, or some combination thereof. In some embodiments, the user may interact directly with an interface (not shown) on SFTD 125, or with another device (e.g. a computer, an in-building controller connected by cable or wirelessly to the SFTD, a remote controller, a portable computing device such as a smartphone running an app). In some embodiments, the SFTD 125 may be pre-loaded with at least some PSFPs (e.g., in firmware), may communicate directly with another device (e.g., a portable device, another connected device, a remote device, or a cloud storage) to receive PSFPs, or some combination thereof.

In various embodiments, the communication link 150 and the communication link 120 may be direct or indirect, and may be wireless, wired, or both. Example communication links include, but are not limited to, short distance communication (e.g., Bluetooth, Bluetooth Low Energy (BLE), Zigby), Wi-Fi, cellular communication (e.g., 2G, 3G, 4G, LTE, 5G), short messaging service (SMS), multimedia messaging service (MMS), serial communication, ethernet link, optical link, or some combination thereof.

FIG. 1B depicts an exemplary symphonic frequency therapy system. For example, the symphonic frequency therapy system may be deployed in the illustrative use case described in FIG. 1A. In this example, the portable device 110 includes a mobile app 160. In some implementations, the portable device 110 may execute the instructions of the mobile app 160 to generate the use interface 115 for user selection.

In some implementations, the mobile app 160 may include instructions to selectively display PSFPs on the portable device 110 for user selection. In the depicted example, the portable device 110 is connected to a recommendation engine 165 and one or more sensors 170. For example, the sensors 170 may include biological sensors capturing biofeedback data related to a physiological condition of the user 105. For example, the biofeedback may include heart rate, heart rate variability (HRV), sleep pattern, EEG (electroencephalogram) signals, breathing pattern, or some combination thereof. In some examples, the sensors 170 may include at least one sensor embodied in a wristband health and fitness tracker. In some examples, the sensors 170 may include at least one sensor embodied in a smartwatch. In some implementations, the mobile app 160 may communicate with the sensors 170 and receives the biofeedback data. In some implementations, the recommendation engine 165 may recommend one or more PSFPs to the mobile app 160 based on data captured by the sensors 170.

In various embodiments, the recommendation engine 165 may be integrated in the portable device 110. For example, the recommendation engine 165 may include at least one module of the mobile app 160. In another example, the recommendation engine 165 may include a separated software installed on the portable device 110. In other examples, the recommendation engine 165 may include a cloud service provided to the mobile app 160 via the Internet. For example, the recommendation engine 165 may implement a subscription service.

The portable device 110 includes user profile 175 in the depicted example. In some examples, the user profile 175 may include metadata related to the user 105. For example, the metadata may include a user's intent to use the device, age, gender, health history, activity history, or some combination thereof (e.g., metadata related to the user 105). In some implementations, when a PSFP recommendation is required, the mobile app 160 may transmit the user profile 175 and the data captured by the sensors 170 to the recommendation engine 165. In one embodiment, the recommendation engine 165 may recommend one or more PSFPs to the mobile app 160 based on the received data. Upon receiving the recommendations, for example, the mobile app 160 may display the recommendations on the interface 115 for user's selection. In various embodiments, the mobile app 160 may use the recommendation engine 165 to advantageously display substantially personalized PSFPs for user selection.

In some implementations, the mobile app 160 may receive particular PSFPs suggested by the recommendation engine 165 based on various behavior and/or characteristics of the user 105. For example, the user profile 175 may include past browsing history, previous activities, internet of things (IoT) connected data regarding user behavior or environment, personal characteristics (e.g., activity level, gender, profession, self-described attributes, height, weight), or some combination thereof. Based on the user profile 175, the recommendation engine 165 may, in some implementations, utilize predetermined and/or customized guidance models to select one or more PSFPs for recommendation.

As an illustrative example, the recommendation engine 165 may suggest one or more PSFPs with “COOL DOWN” effect for a post-exercised user. For example, the “COOL DOWN” PSFP may be (automatically) indicated in response to receiving signal(s) from a location tracker of the portable device 110 indicating that the user has visited a gym, and/or receiving signal(s) from a smartwatch indicating that the user 105 has exercised. In some embodiments, the recommendation engine 165 may suggest relevant wellness promotion PSFPs when a body monitoring app indicates a physical disorder such as, by way of example and not limitation, elevated heart rate, oxygenation, and/or temperature. In some examples, the recommendation engine 165 may suggest a deep sleep PSFP when device usage indicates an abnormally late working hours, an abnormally short or disturbed sleeping time, and/or location tracking indicates that the user 105 is sleeping away from home. In another example, the recommendation engine 165 may suggest a relaxation PSFP after the user 105 has engaged a connected device with abnormal levels of intensity. In another example, the recommendation engine 165 may suggest a dental health PSFP after detecting that the user 105 has just finished a dentist appointment. In another example, the recommendation engine 165 may suggest a mold treatment PSFP when the user 105 searches (e.g., through a search engine, through a virtual assistant) for mold remedies. In another example, the recommendation engine 165 may suggest seasonally relevant PSFPs based on time of year.

In some embodiments the recommendation engine may, by way of example and not limitation, include one or more machine learning models. The model may, for example, operate on signals corresponding to metadata, environmental data, and/or biofeedback data to generate signal(s) indicative of suggested and/or recommended PSFPs. The PSFP suggestions and/or recommendations may, for example, be generated as a function of historical data of the user 105 and/or other users. For example, historical data from other users may include PSFPs selected by other users corresponding to metadata, environmental data, and/or biofeedback data from other users. The recommendation engine may, for example, generate recommendations and/or suggestions based on historical user selections.

The recommendation engine may, for example, generate recommendations and/or suggestions as a function of historical selections and/or responses compared to user attributes. A machine learning model may, for example, generate recommendations and/or suggestions based on the user metadata, environmental data, and/or biofeedback data as compared to (historical) data from other users. For example, the model may be applied to a user's metadata (e.g., 35 years, female), environmental data (e.g., high pollen count), biofeedback data (e.g., user breathing indicates allergic response), and generate recommendation and/or suggestion signal(s) based on PSFPs corresponding to other users with similar attributes. For example, the PSFPs corresponding to other users may include, by way of example and not limitation, PSFPs selected by other users, PSFPs recommended by other users, PSFPs applied to other users, PSFPs correlated to (predetermined) positive responses when applied to other users, or some combination thereof.

The recommendation engine may, for example, generate recommendations and/or suggestions as a function of historical and/or current responses. Responses may, for example, include changes in biofeedback data (e.g., positive and/or negative changes). For example, a deep sleep PSFP may be suggested based on other users experiencing improved sleep (e.g., as determined by biofeedback data indicating longer, less interrupted, and/or predetermined desirable sleep state attributes as compared to historical sleep data). Responses may include, for example, biofeedback data indicated improved sleep, HRV, oxygenation, blood pressure, or some combination thereof. Responses may, for example, include solicited and/or unsolicited user responses. User responses may, for example, include responses to survey questions (e.g., did this PSFP help you? Did you experience improved ______ after using this PSFP?). User responses may, for example, include user reviews.

FIG. 1C depicts an exemplary method of selecting PSFP(s) for generating a symphonic PEMF. In a method 180, a portable device (e.g., the portable device 110) receives user data in a step 181. The user data may include, by way of example and not limitation, biofeedback data, environmental data, user metadata, or some combination thereof. A user profile (e.g., the user profile 175) is generated in a step 182. The user profile may, for example, be generated by the portable device. In some embodiments the user profile may, for example, be generated, by a remote system (e.g., central server) operably coupled to the portable device, in response to signals (e.g., user data) from the portable device).

A sensor signal(s) is received in a step 183. The sensor signal may, for example, be received from at least one of the sensors 170. The sensor signal may, for example, correspond to a request for a SPEMF from a user, to a trigger for generating a SPEMF (e.g., predetermined trigger), to environmental data, to biofeedback data, or some combination thereof.

A recommendation request is generated by the portable device in a step 184. The recommendation request may, by way of example and not limitation, include the user profile and/or data corresponding to the sensor signal(s). The recommendation request may, for example, be generated according to at least one predetermined request format. The request may, for example, be generated and transmitted as a request file according to at least one predetermined request format. The request may be transmitted to a recommendation engine (e.g., the recommendation engine 165).

When the recommendation engine receives a recommendation request (e.g., from the portable device), in a decision point 185, then a recommendation signal is generated in a step 186. For example, the recommendation signal may include (identifiers of) one or more PSFPs. The recommendation signal may, for example, be generated based on the recommendation request (e.g., as a function of the user profile, as a function of the sensor signal(s)).

When a recommendation signal is received by the portable device in a decision point 187, and a user selection of a PSFP is received in a decision point 188, then a corresponding recommended PSFP is retrieved (e.g., from the remote data store 155) in a step 189. The portable device may, for example, generate a user interface(s) in response to the recommendation signal. The user interface may, for example, indicate one or more of the recommended PSFP(s) identified in the recommendation signal. The user interface may, for example, be configured to prompt a user for a selection input (e.g., selecting one of multiple recommended PSFPs) and/or approval input (e.g., approving a recommended PSFP).

Once the recommended PSFP(s) is received in the step 189, then a signal(s) is generated in a step 190 and transmitted to an SFTD (e.g., the SFTD 125). The signal may, for example, include a PSFP identifier. The SFTD may, for example, activate a pre-loaded PSFP already stored in local memory of the SFTD in response to the signal from the portable device. The SFTD may, for example, retrieve a corresponding PSFP(s) (e.g., from the remote data store 155) in response to the signal from the portable device. The signal may, for example, include a PSFP (e.g., metadata configured to define a SPEMF).

When the SFTD receives a signal identifying and/or including a PSFP from the portable device in a decision point 191, then the SFTD generates signal(s) in a step 192 configured to drive a coupled coil(s) (e.g., the 140) to generate a time-varying SPEMF corresponding to the PSFP.

FIG. 2A depicts an exemplary interface for selecting symphonic frequency profiles. A portable device 205 displays an interface for a user to browse and select various PSFPs. Illustrative “HEALTH” category 210 includes icons indicating one or more PSFPs which may be associated with, by way of example and not limitation, cranial or cognitive health (icon 212), cardiovascular health (icon 214), operation healing health (icon 216), and dental health (icon 218). Each icon may, for example, represent a single PSFP, or may represent a sub-category containing further categories or individual PSFPs. For example, selection of the operation healing health icon 216 may select a post-surgery PSFP generally applicable after surgery or, when selected, may cause portable device 205 to present further PSFPs which may, for example, be designed for bone fracture, soft-tissue surgery, or even more specific therapeutic intents.

Similarly, illustrative “WELLNESS” category 220 includes icons which may be associated with exercise (icon 222) and rest (icon 224). Exercise (icon 222) may, for example, select a subcategory containing PSFPs directed to various pre- or post-exercise therapy, activity-specific PSFPs (e.g., running, walking, jogging, climbing, cross-fit, cardio, basketball, tennis, golf, soccer, yoga). Rest (icon 224) may, for example, select a subcategory containing PSFPs directed to various pre- or post-rest therapies, for example, relaxation promotion, deep sleep promotion, exhaustion therapy, and morning post-sleep therapy.

The “MORE” button 230 may cause portable device 205 to present further categories to the user. For example, when the “MORE” button 230 is selected, the interface may further receive a query from a user to find PSFPs according to a current intent of the user. The interface may suggest PSFPs according to current and/or past interactions and/or activities of the user. In one implementation, the interface may transmit the user intent and/or other metadata to the recommendation engine 165 to find additional PSFPs to be displayed for user selection.

FIG. 2B depicts an exemplary interface for initiating generation of a selected symphonic frequency profile. The depicted illustrative interface presented on portable device 205 may, for example, be presented when a user selects icon 212 representing headache therapy. A more detailed description of the selected PSFP is presented to the user, including icon 240, descriptive title 242, detailed description 244, and activation command button 246. A user suffering from a headache may, for example, determine to use the PSFP based on the description 244. The user then may engage button 246, which causes the portable device 205 to initiate command operations to cause a SFTD to generate electrical signals transduced by attached coil into the predetermined symphony of pulsed electromagnetic field (EMF) sinusoidal frequencies according to the parameters of the selected PSFP.

FIG. 2C depicts an exemplary interface for selecting symphonic frequency profiles. A portable device 248 displays an interface for a user to browse and select various PSFPs. Illustrative “PERFORMANCE” category 250 includes icons indicating one or more PSFPs which may be associated with, by way of example and not limitation, cognitive performance optimization (icon 252), harmony balance (icon 254), energy boost (icon 256), workout preparation (icon 258), and endurance (icon 260). Each icon may, for example, represent a single PSFP, or may represent a sub-category containing further categories or individual PSFPs.

The “MORE” button 264 may cause portable device 248 to present further categories to the user. The interface may further receive a query from a user to find PSFPs according to a current intent of the user. The interface may, for example, suggest PSFPs according to current and/or past interactions and/or activities of the user. Other categories may include, for example, “WELLNESS,” “OPTIMIZATION,” “FOCUS,” and “RECOVERY.”

FIG. 2D depicts an exemplary interface for initiating generation of a selected symphonic frequency profile. The depicted illustrative interface presented on portable device 248 may, for example, be presented when a user selects icon 252 representing cognitive performance optimization. A more detailed description of the selected PSFP is presented to the user, including icon 268, descriptive title 270, detailed description 272, and activation command button 274. A user wishing, for example, to sharpen and enhance the mind and release mental tension may determine to use the PSFP based on the description 272. The user then may engage button 274, which causes the portable device 248 to initiate command operations to cause a SFTD to generate electrical signals transduced by an attached coil into the predetermined symphony of pulsed EMF sinusoidal frequencies according to the parameters of the selected PSFP.

FIG. 3 depicts an exemplary symphonic frequency therapy device (SFTD 305). The SFTD 305 includes a Bluetooth receiver panel 310, a power status indicator 315, a coil status indicator 320, and a PSFP status indicator 325. The SFTD 305 may be provided with a rigid or semi-rigid case, and a solid front panel. The Bluetooth receiver panel 310 may be a partially translucent panel. The panel may advantageously permit communication signals to pass between a user's portable device and a Bluetooth communication circuit in the SFTD 305 to receive, for example, a command from the portable device to generate a specific PSFP selected by a user, and/or to receive a PSFP from the portable device. The status indicators may allow a user to view a current status of the SFTD 305 in general, and various functions and components thereof. For example, power status indicator 315 may indicate when power is provided to the device (e.g., by being continuously lighted). Coil status indicator 320 may indicate when the coil is properly connected and available (e.g., by being continuously lighted). PSFP status indicator 325 may indicate, for example, when a PSFP is being downloaded (e.g., by displaying a blinking light, by displaying a light of a specific color, or both). The PSFP status indicator 325 may indicate when the coil is being actively driven to produce symphonic sine signals according to a currently selected PSFP (e.g., by displaying a continuous light, by displaying a light of another specific color, or both).

The SFTD 305 may implement parameters of a currently selected PSFP through various circuitry to generate a set of signals to a handheld solenoidal coil 340 to produce the desired symphonic sine signal set at frequencies, amplitudes, and/or phasing specified by the PSFP. Signals pass through a cable 335, connected to the SFTD 305 by a connector 330. The cable 335, in this example, is connected to handheld solenoidal coil 340, which produces a toroidal shaped PEMF symphony defined by the PSFP. In some embodiments, when a frequency set plays, a light on the coil 340 may illuminate and/or animate based on the symphonic frequencies. In various embodiments, the SFTD 305 may drive the handheld solenoidal coil 340 to generate a time-varying symphonic PEMF according to a frequency set defined by the PSFP and including at least 50 frequencies of at least 4 different orders of magnitude and defining a range of at least 10¹-10⁴ Hz.

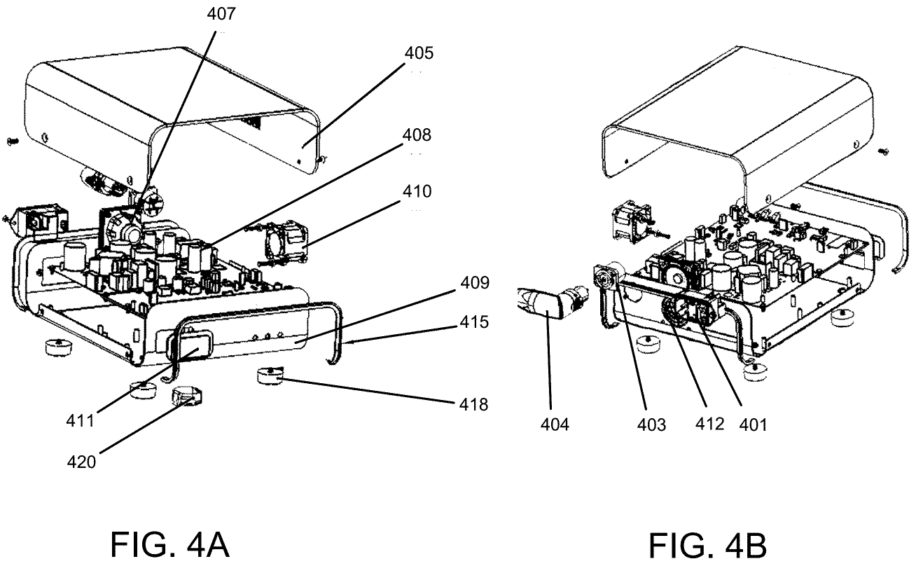

FIG. 4A depicts a first exploded view of an exemplary symphonic frequency therapy device, and FIG. 4B depicts a second exploded view of the exemplary symphonic frequency therapy device of FIG. 4A. A SFTD chassis bottom 409 receives driver board 408. In some embodiments, a cooling fan 410 may be provided. The front of the chassis bottom 409 is provided with a Bluetooth cover 411. A feet 418 and lens 420 are disposed on the underside of the chassis bottom 409. In some embodiments, a seal 415 (front and back) may be provided to provide sealing between the chassis bottom 409 and a chassis top 405. In some embodiments, tolerances may be sufficiently tight to seal undesired solids and fluids out without a seal component. A speaker 407 may provide audio feedback to a user, for example, at startup, shut down, successful connection of a coil, ready to receive a PSFP, successful receipt of a PSFP, beginning driving a connected coil according to a current PSFP, troubleshooting, or some combination thereof. On the rear of the chassis bottom 409 is mounted a power entry module 401, a chassis coil cable connector 403, and a speaker cover 412. Coil cable (not shown) is terminated with a right-angle connector 404, which is configured to connect with the chassis coil cable connector 403. The SFTD may drive a coil (not shown) according to parameters determined by a PSFP selected by a user and, for example, pre-loaded in firmware, received from a portable device or a remote data store, or a combination thereof. The coil, which may be a handheld solenoidal coil may, thus, transduce electrical signals received from the SFTD into a predetermined symphony of PEMF sine waves according to the parameters of the PSFP.

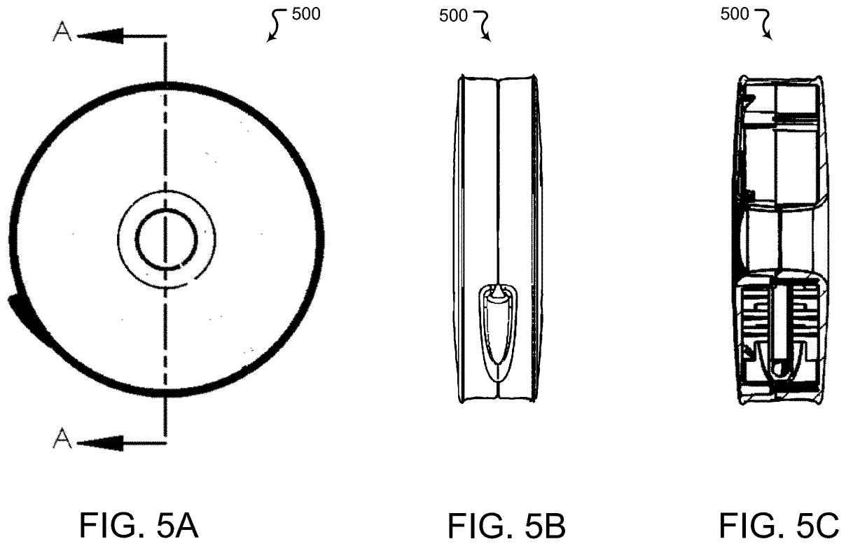

FIG. 5A depicts a top plan view of an exemplary solenoidal coil 500 which transduces electric signals from a SFTD into a toroidal shaped electromagnetic field of symphonic pulsed sine waves according to a predetermined symphonic frequency profile. FIG. 5B depicts a side elevation view of the exemplary solenoidal coil 500 of FIG. 5A. FIG. 5C depicts a cross-section A view of the exemplary solenoidal coil 500 of FIG. 5A.

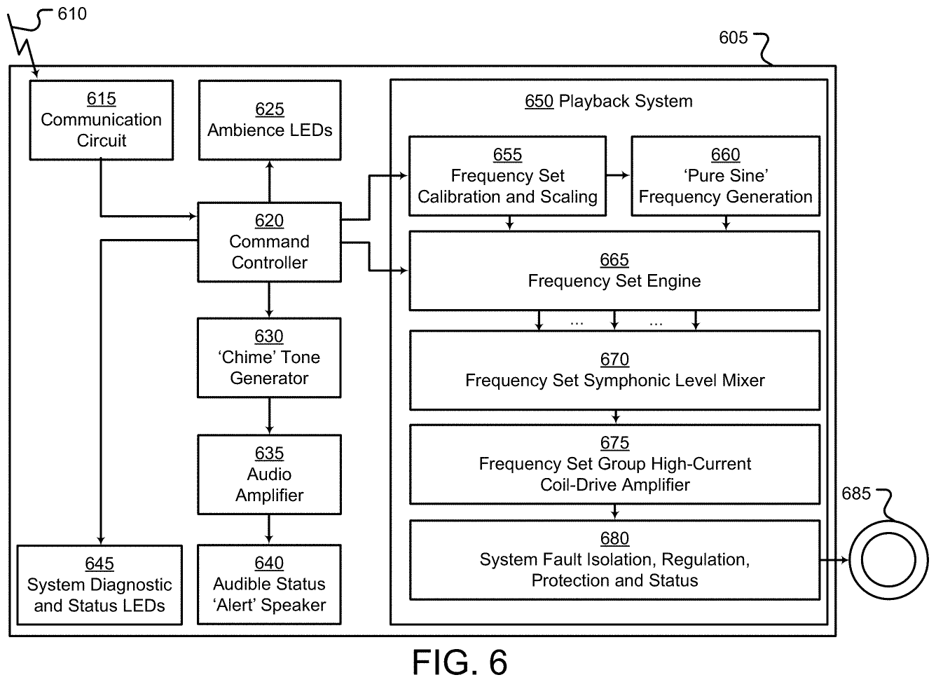

FIG. 6 depicts a block diagram of an exemplary symphonic frequency therapy device (SFTD 605). The SFTD 605 receives a communication 610 into a communication circuit 615, which is operatively connected to a command controller 620. A communication 610 may be, for example, a PSFP, or a command to generate a symphony of sine waves according to a currently selected PSFP. Generation may include, for example, mixing individual sine waves or groups of sine waves. The communication circuit 615 may include, for example, a Bluetooth receiver and/or transceiver, an ethernet port, and/or a Wi-Fi receiver. In various implementations, the command controller 620 may include an integrated circuit. In some examples, the command controller 620 may be provided with one or more processors (e.g., microcontrollers) configured to receive a PSFP through the communication circuit 615 and direct other circuits according to the parameters contained in the PSFP to generate the predetermined symphony of sine waves as prescribed by the PSFP.

The command controller 620 controls system diagnostic and status LEDs, for example, according to current states of the SFTD 605 or components thereof. The command controller 620 controls ambience LEDs 625 according to, for example, additional lighting parameters contained in the PSFP. The command controller 620 sends command inputs according to the current PSFP to a playback system 650, which generates a signal to drive a coil 685. Specifically, the command controller 620 provides inputs to a frequency set calibration and scaling circuit 655 and a frequency set engine 665. A pure sine frequency generation circuit 660 generates a base sine wave according to the base frequency, amplitude, and phase parameters of the PSFP received from the frequency set calibration and scaling circuit 655 provides an input. The frequency set engine 665 receives the base pure sine wave generated by pure sine frequency generation circuit 660, and sets additional symphonic sine waves specified in the PSFP according to the frequency set calibration and scaling circuit 655. The resultant output of the frequency set engine 665 are the individual component waves specified in the PSFP selected by the user, as indicated by the plurality of outputs separated by ellipses, which may represent additional (or fewer) sine waves.

A frequency set symphonic level mixer 670 receives the output individual sine waves and combines them to form a single symphonic frequency wave output. This output is amplified to drive the coil 685 by a frequency set group high-current coil-drive amplifier 675. The resultant amplified output is conditioned by a system fault isolation, regulation, protection, and status circuit 680. The final output provides an electrical signal transduced by the coil 685 to produce a PEMF prescribed by the PSFP selected by the user according to a therapeutic need or desire of a user.

The command controller 620 further sends a (command) signal to a ‘chime’ tone generator 630. The ‘chime’ tone generator 630 generates, in response to receiving the signal from the command controller 620, a signal to an audio amplifier 635. In response to receiving the signal from the ‘chime’ tone generator 630, the audio amplifier 635 provides a drive signal to an audible status ‘alert’ speaker 640 (e.g., the speaker 407). The command controller 620 further generates a command signal to a system diagnostic and status LEDs 645. The LEDs 645 may, for example, generate visual indicia in response to the command signal (e.g., corresponding to a status of the SFTD 605, the playback system 650, and/or some component(s) thereof).

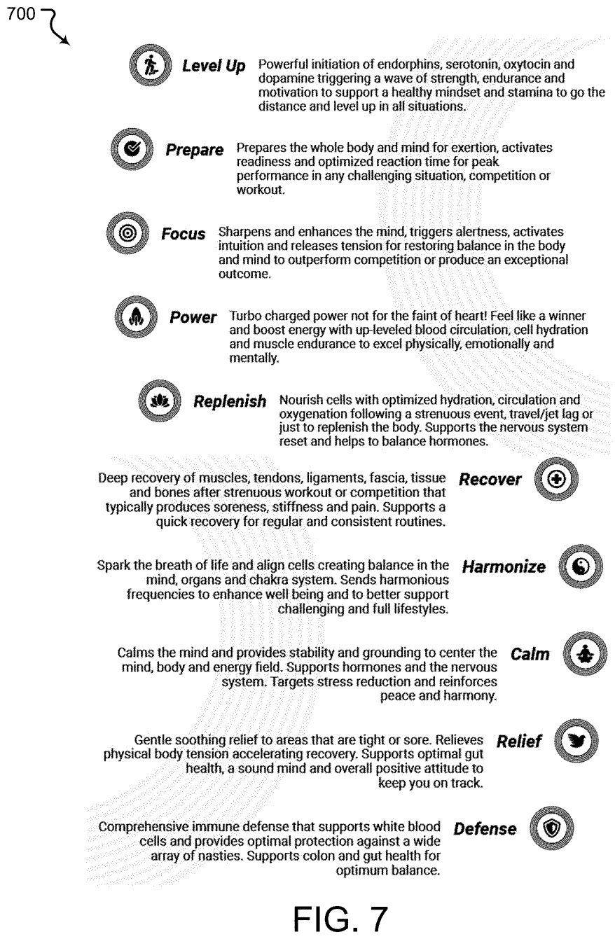

FIG. 7 depicts exemplary predetermined symphonic frequency profiles table 700. The table 700 depicts exemplary PSFP names and descriptions that may be presented to users to select according to a desired optimization or performance effect. For example, each associated PSFP may be sufficient to cause a unique SPEMF to be produced that is associated with effects as described in the table 700.

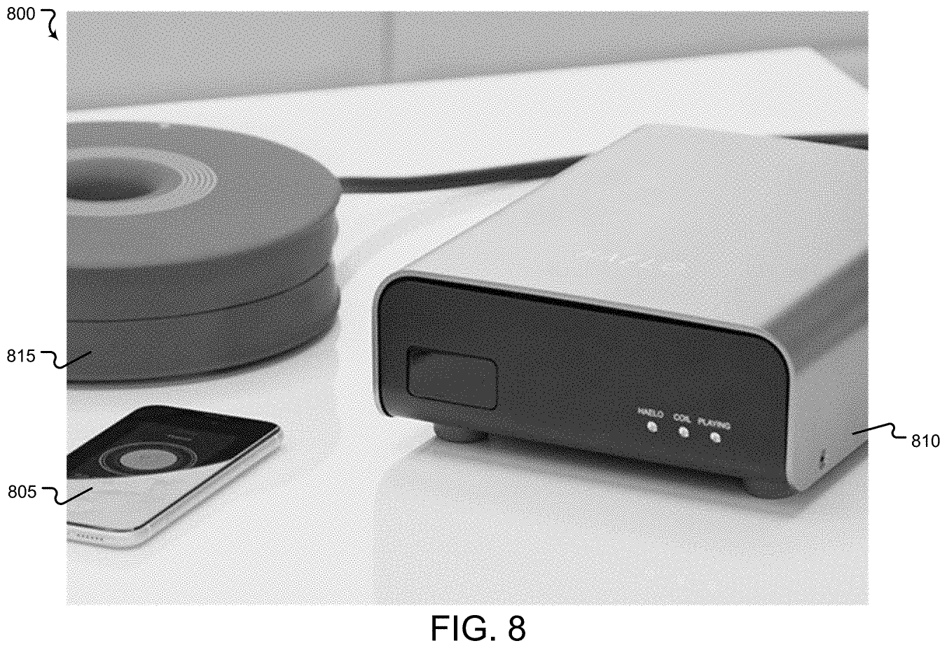

FIG. 8 depicts an exemplary implementation of a symphonic frequency therapy device. In this example, a symphonic pulsed frequency therapy system 800 includes a SFTD 810 connected to a coil 815. The system 800 further includes a smartphone 805 that is running an app to communicate commands to the SFTD 810. For example, the app may command the SFTD 810 to select a PSFP for generating an associated PEMF.

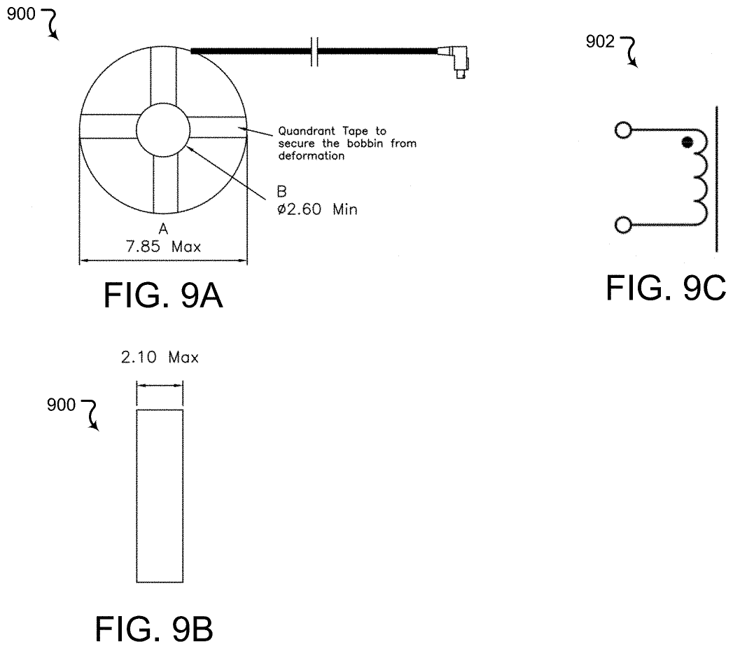

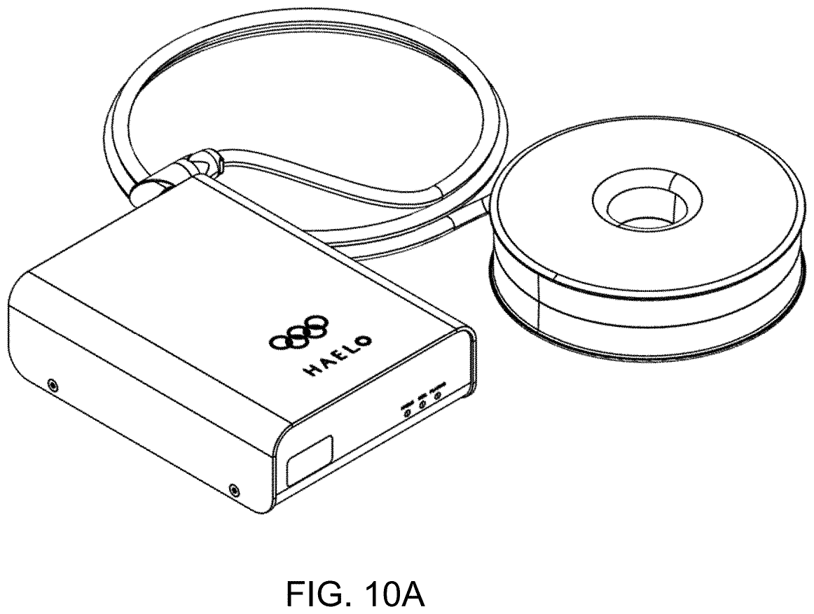

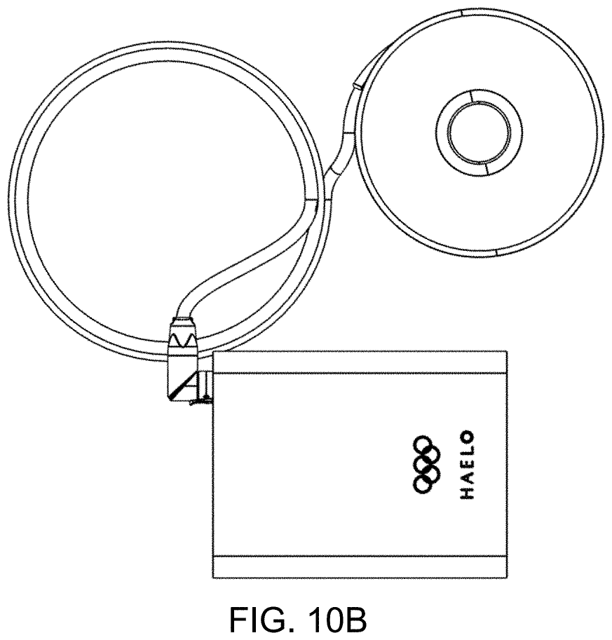

FIG. 9A depicts a top view of an exemplary implementation of a solenoidal coil. FIG. 9B depicts an end view of the exemplary solenoidal coil of FIG. 9A. FIG. 9C depicts a schematic of the exemplary solenoidal coil of FIG. 9A. In the depicted implementation, coil 900 is depicted without an outer casing. Illustrative coil 900 has a diameter of approximately 7.85 inches, an inner aperture diameter of approximately 2.60 inches, and a thickness of approximately 2.10 inches. In some implementations, the weight of the coil 900 may be substantially less than 6 lbs. The actual dimensions of the depicted implementation will be determined according to the actual coil material used (e.g., conductor thickness) and actual winding (e.g., actual numbers of windings). The coil cable's length may be 120 inches long, and the cable may be IEC 53 (RVV) 2×2.5 mm sheathed flexible cable. Dimensions may be within +/−0.010 inches. The connector at the end of the cable may be, for example, a Neutrik NL2FX. As shown by schematic 902, the coil may be a 333-turn coil of 11 AWG heavy poly nylon (HPN) round magnet wire rated for an operating temperature of 155° C. The coil may have a typical open circuit conductance of 17.3 mH at 1 kHz and 1V, and a typical direct current resistance of 1.01 Ohms.

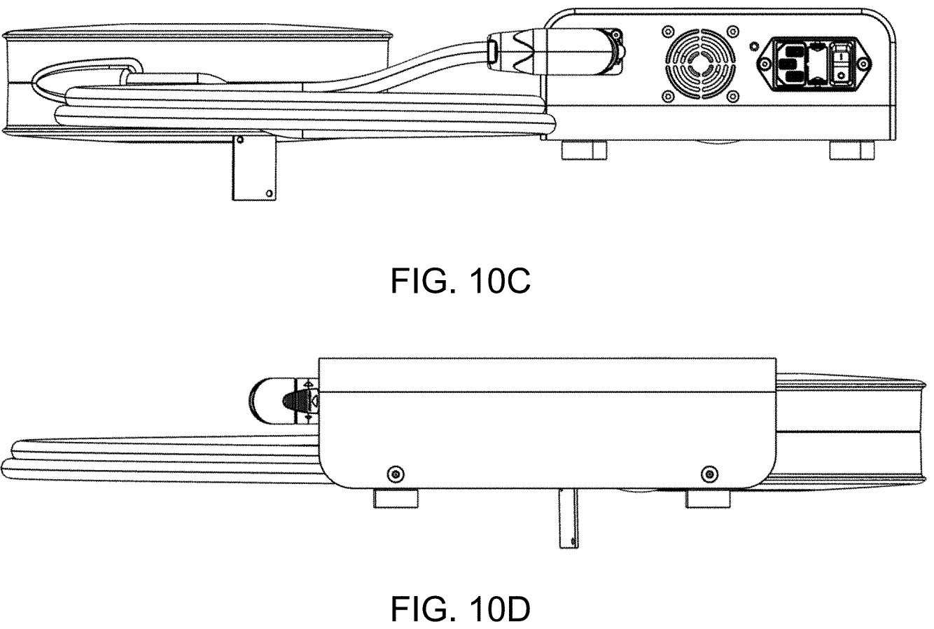

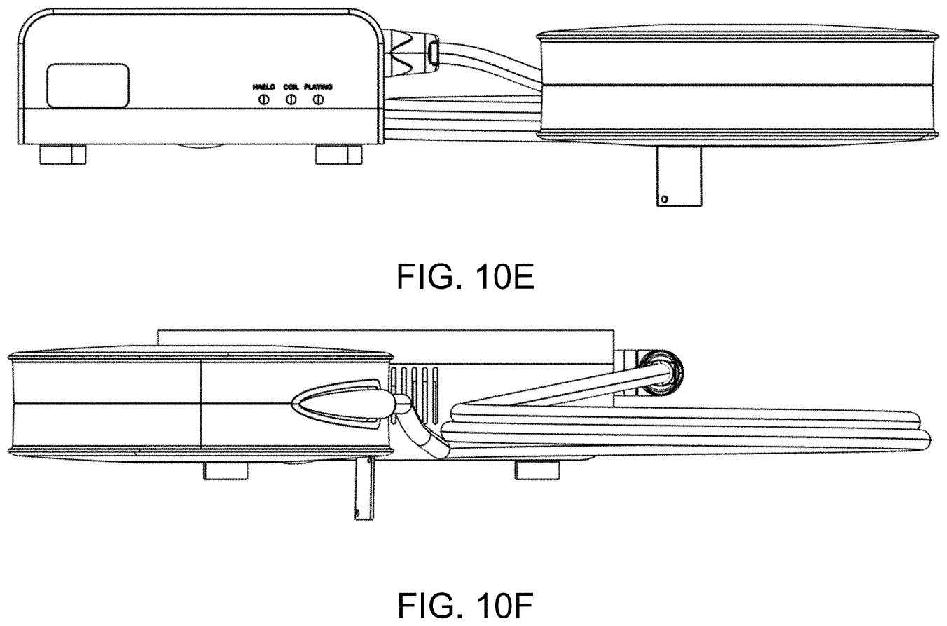

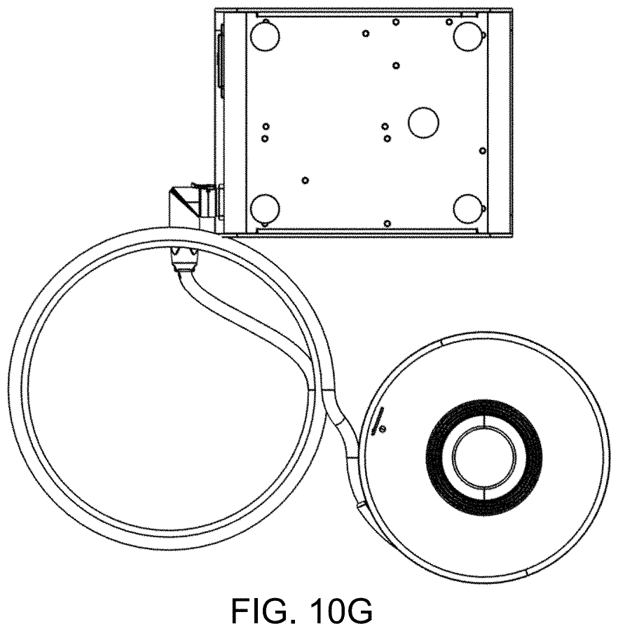

FIG. 10A, FIG. 10B, FIG. 10C, FIG. 10D, FIG. 10E, FIG. 10F, and FIG. 10G depict a perspective view, a top view, a rear view, a left-side view, a front view, a right-side view, and a bottom view of an exemplary PEMF therapy system.

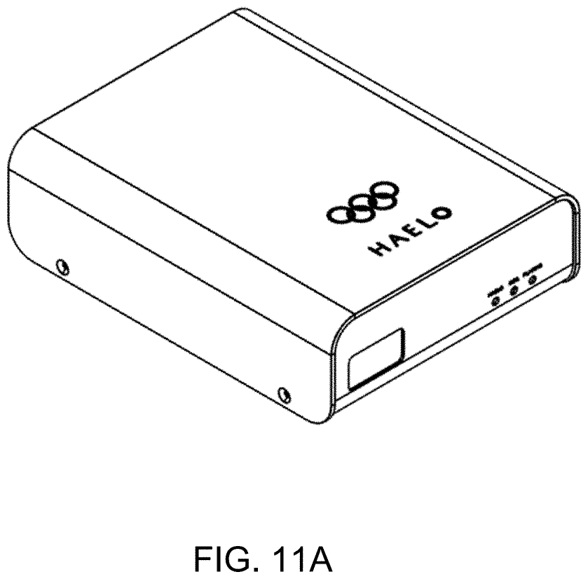

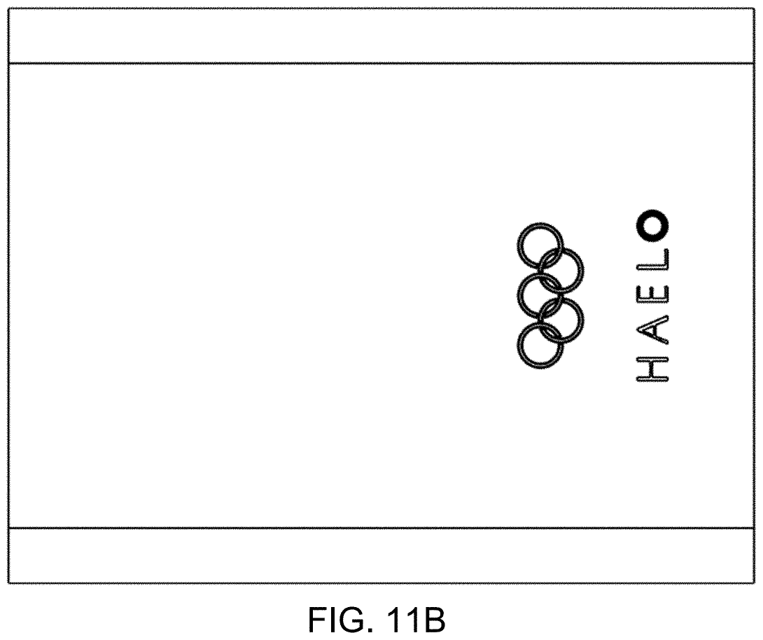

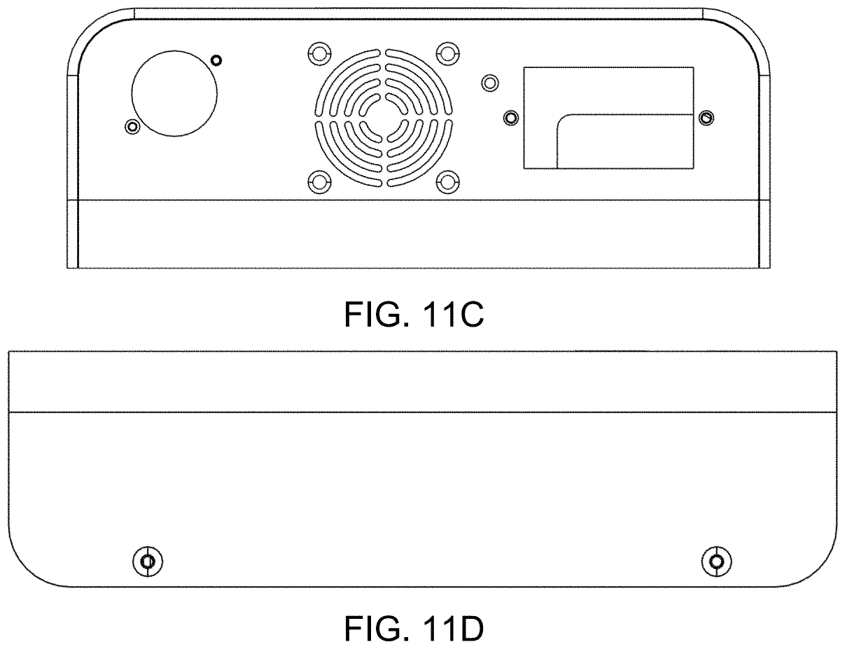

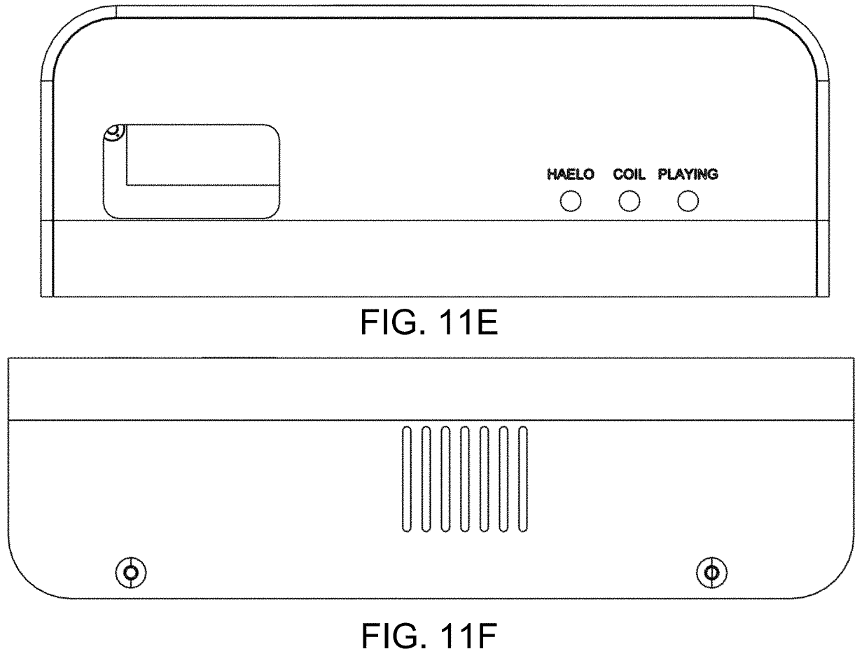

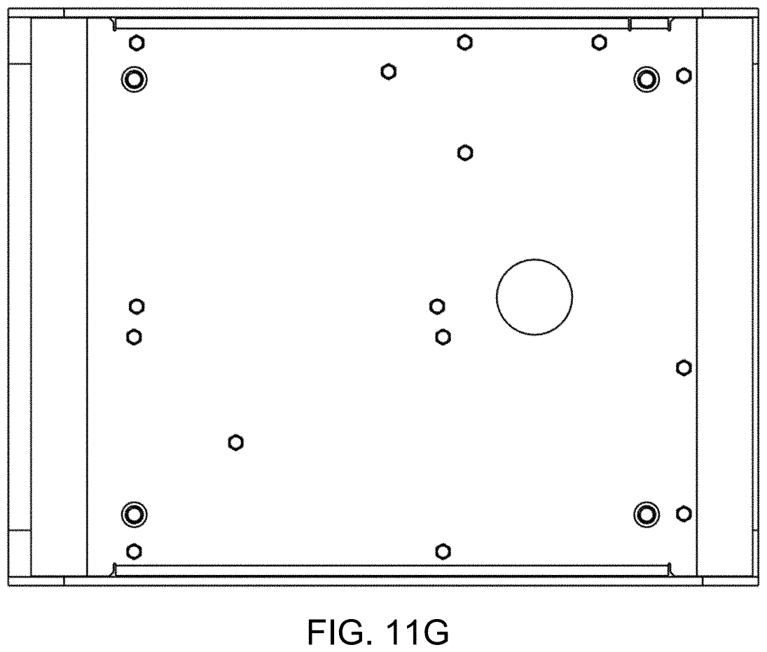

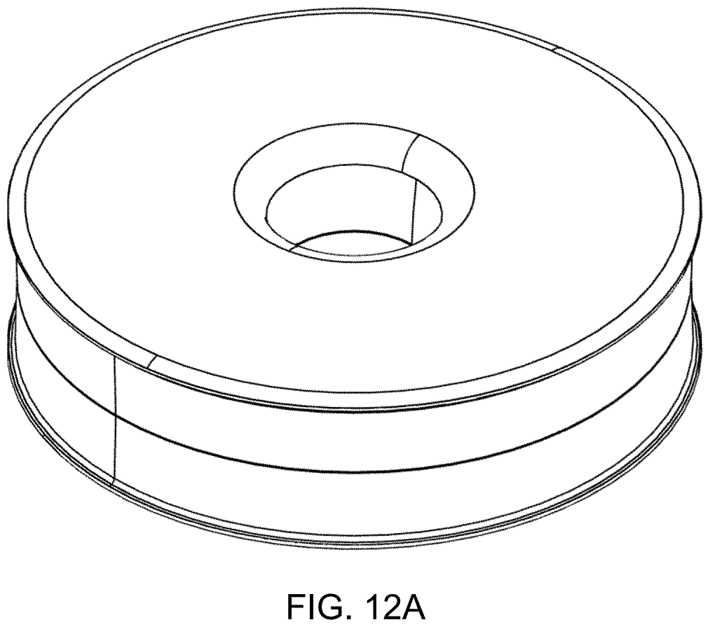

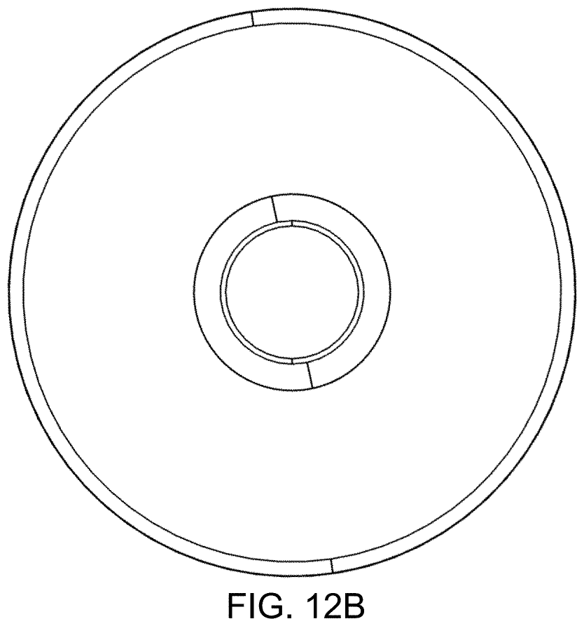

FIG. 11A, FIG. 11B, FIG. 11C, FIG. 11D, FIG. 11E, FIG. 11F, and FIG. 11G depict a perspective view, a top view, a rear view, a left-side view, a front view, a right-side view, and a bottom view of an exemplary symphonic frequency therapy device.

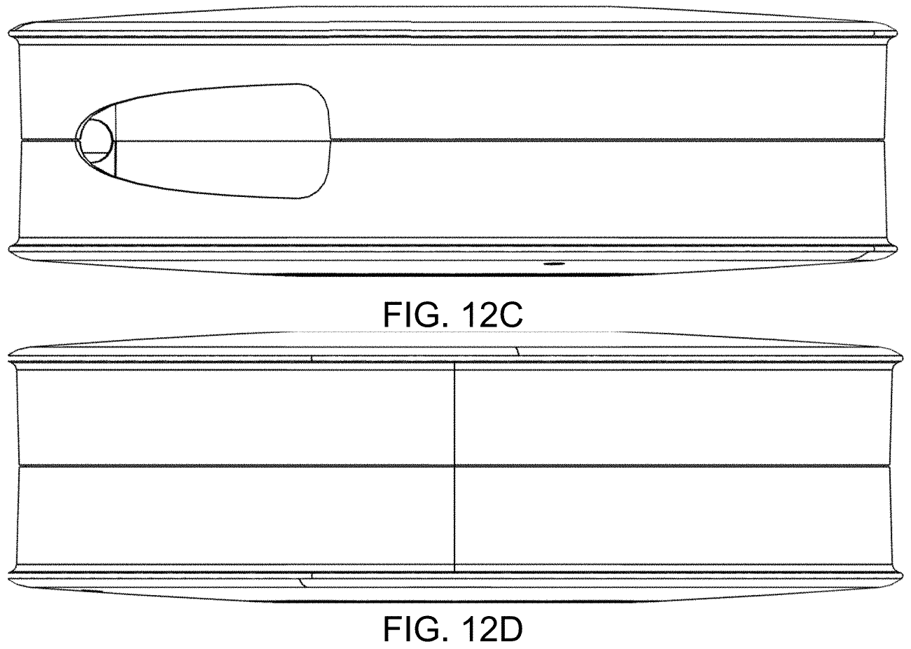

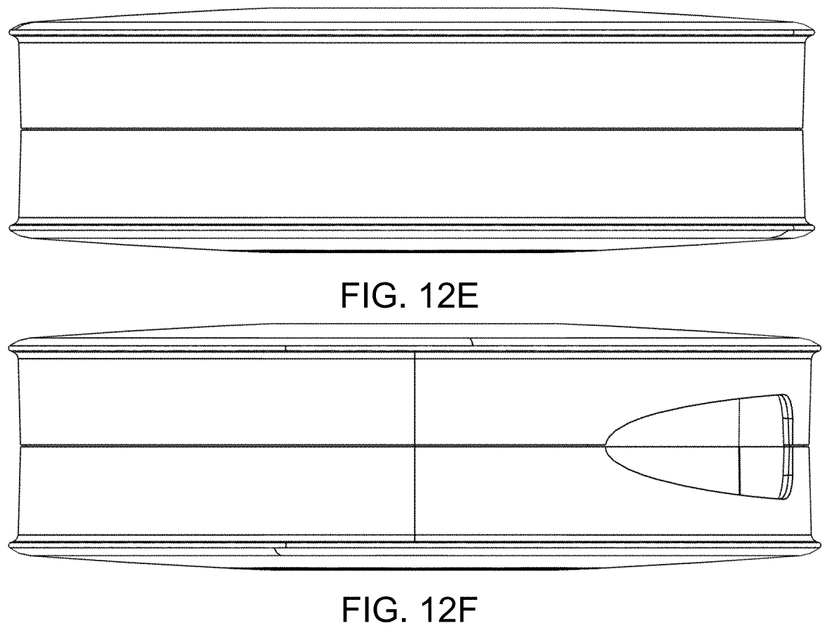

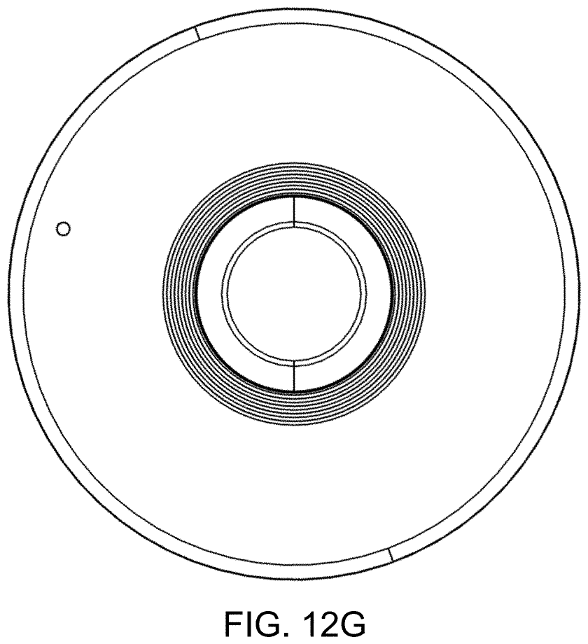

FIG. 12A, FIG. 12B, FIG. 12C, FIG. 12D, FIG. 12E, FIG. 12F, and FIG. 12G depict a perspective view, a top view, a rear view, a left-side view, a front view, a right-side view, and a bottom view of an exemplary handheld solenoidal coil.

Although various embodiments have been described with reference to the figures, other embodiments are possible. For example, in some embodiments, one or more of the PSFP parameters may be time-variable. A particular PSFP may include parameters sufficient to create a unique time-varying symphony of pure sine wave, generating a time-varying symphonic PEMF (SPEMF).

In some embodiments, SFTDs, coils, and associated PSFPs may be strictly non-medical and non-therapeutic with respect to medical therapies. Such embodiments may, for example, be directed to mental, physical, or emotional wellness, optimization, performance, or focus, or some combination thereof. In some embodiments, SFTDs, coils, and associated PSFPs may provide medical therapies directed to treating, for example, illness and trauma.

In some embodiments, a symphonic frequency therapy system is provided with a plurality of controllers, a plurality of SFTDs and coils, or some combination thereof. Such embodiments may be useful, for example, in multi-patient therapy facilities (e.g., chiropractic offices, wellness clinics, physical therapy clinics, hospitals, and physicians' clinics). For example, a plurality of SFTDs and coils may be installed in a specially purposed locale. In some examples, the plurality of SFTDs and coils may transmit PEMFs 24 hours a day to promote calming, relaxing, and harmonization within the specialized locale.

In some embodiments, a user selecting a PSFP may be different from the user receiving the resulting SPEMF. For example, a practitioner, at a remote location from the user, may choose a prescribed PSFP according to a patient's symptoms, environment, or condition; in response to the prescribed PSFP information being transmitted (e.g., via the Internet) to an SFTD local to the patient, the SFTD may administer SPEMF to the patient according to the prescribed PSFP. Similarly, a user may choose a PSFP to generate a SPEMF for a non-human subject, such as, for example, a veterinarian for an animal, a homeowner or treatment practitioner for home treatment use, fishermen for fishes in fisheries, or a horticulturist for plants in a greenhouse. For example, energy may be advantageously infused by the SFTD and the coils using various PSFPs for promoting harmonization, optimization, nutrient infusions, or a combination thereof.

In various embodiments, an SFTD may be configured to receive one or more interchangeable coils. In various implementations, a coil may be conformable to some portion of the body. A coil may be provided with a gel and/or other conformable coating to, for example, increase comfort, fit, conductance of the symphony of sinusoidal waves, or a combination thereof. A coil may be provided in a predetermined shape optimized to fit one or more body part such as, by way of example and not limitation, a limb (e.g., leg, arm, hand, foot), joint (e.g., knee, shoulder, elbow, wrist), or face. Some embodiments may be provided with a kit of coils optimized for different uses. Some embodiments may be optimized for a particular application. For example, some implementations may include a SFTD preloaded with face and beauty related PSFPs (e.g., such as discussed elsewhere herein), and may be provided with one or more coils optimized, for example, for application to the face or for other beauty-related applications.

In an exemplary embodiment, a coil unit may be (releasably) coupled to an SFTD. The coil unit may, for example, be configured to fit a body portion. The body portion may, for example, include a face. The coil unit may, for example, be disposed in a housing configured to fit to and/or be molded to fit a human face. The coil unit may, for example, include multiple toroidal coils. Each coil may, for example, generate a symphonic PEMF according to the PSFPs based on signal(s) received from the SFTD. The housing may, for example, receive the coil(s). The housing may, for example, include an interface element shaped to and/or shapeable to the body portion. The interface element may, for example, be releasably coupled to the housing. The interface element may, for example, be custom fitted to a specific user (e.g., molded, 3D-printed, heat-shaped) to a user's body portion (e.g., face, limb, torso). The interface element may, for example, be coupled to the housing such as, for example after being custom fitted. Accordingly, various embodiments may advantageously be custom fitted to a user. Various embodiments may advantageously be adaptable to be custom fitted to multiple users by one or more custom fitted releasably coupled interface elements.

In various embodiments, the SFTD may generate and amplify frequency sets (as defined by PSFPs) that contain 150 or more individual frequencies per frequency set. Some implementations may generate symphonic PEMFs according PSFPs having frequencies between 3 hertz (Hz) and 11,875 Hz. Some implementations may be designed and configured to primarily operate below 5000 Hz.

In some embodiments the coil and SFTD are configured such that the coil does not generate appreciable heat, or coil vibration. Accordingly, various embodiments may advantageously achieve increased coil longevity. Various embodiments may achieve greater user comfort. Accordingly, various embodiments may advantageously achieve increased user compliance. In some such embodiments the PSFPs may be adapted and/or selected specifically to be compatible with the circuitry of the SFTD to minimize heat and vibration.

In an illustrative implementation, a low resistance (e.g., 1.01 Ohm) solenoidal coil is provided having, for example, 333 wraps (winds) of aluminum coated wire. The coil is driven by a SFTD to provide an accurate output of symphonic PEMFs, according to selected PSFPs, as a toroidal fountain cascade into the body or subject. In some embodiments, the output of the frequencies through the coil may be measured in microTeslas (μT).

In some implementations, the SFTDs and the coil may be battery powered. For example, the SFTDs and the coil may include a (re)chargeable battery. In some examples, the battery may advantageously enable the use of the SFTD and the coil in location without AC power. In some implementations, a SFTD may be configured to drive multiple coils. In some examples, a single SFTD may be configured to drive coils located in different rooms of a building. In some implementations, the SFTDs and the coil may be integrated into a single unit. For example, the SFTDs and the coil may be embedded into furniture. The SFTDs and the coil may be embedded, by way of example and not limitation, into a bed, a sofa, a car seat, or some combination thereof. In some examples, the SFTDs and the coil may be embedded into wearable gadgets. For example, the SFTDs and the coil may be embedded into face masks, necklace, bracelets, headbands, brooches, or some combination thereof. In some embodiments a coil may, for example, be releasably coupled to a desk, table, and/or other work area.

In some embodiments the SFTD and/or coil may be (automatically) operated based on predetermined profiles. For example, a user may activate an automatic optimization model (e.g., by activating a subscription). One or more sensors may detect user activity, user parameters (e.g., biofeedback data), and/or environmental parameters. PSFP(s) may, for example, be automatically selected as a function of signal(s) received from the sensor(s). The PSFP(s) may, for example, be automatically activated such that the SFTD drives the coil(s) to generate symphonic PEMFs according to the activated PSFP(s). Accordingly, a user may advantageously receive custom selected SPEMFs based on, for example, current activities (concentration, working, exercising, resting). In some embodiments multiple coil(s) and/or SFTD(s) may, for example, be distributed in multiple locations. A controller (e.g., a central controller connected to a portable computing device such as a smartphone and/or smartwatch, a controller embodied in a portable computing device) may cause specific SFTD(s) and/or coil(s) to be activated based on current parameters. The current parameters may, for example, include user location (e.g., detected by GPS, motion detection, heat detection, biometric recognition).

In some embodiments, for example, a controller may include a geolocation module. The geolocation module may, for example, include GPS circuit(s). The controller may select and/or activate specific PSFP(s) according to, for example, predetermined and/or dynamic geofence boundaries. In an illustrative example, a (predetermined) geofence may be stored corresponding to a gym. A PSFP corresponding to pre-exercise may, for example, be activated (e.g., in a portable SFTD and handheld coil) in response to the user entering the geofence corresponding to the gym. A PSFP corresponding to cardio exercise may, for example, be activated in response to biofeedback (e.g., heart rate, breathing pattern, blood pressure, temperature) indicating the user is performing cardio exercise while in the geofence corresponding to the gym. In a second illustrative example, a geofence may be (predetermined) corresponding to a user's home. PSFPs corresponding to relaxation may, for example, be selected and/or activated when the geofence is entered (e.g., according to signals received from a portable computing device) by the user during a predetermined time period (e.g., between 4 PM-7 PM). In some embodiments, a (machine learning) model may automatically define geofences, associated conditions (e.g., time, biofeedback), and/or associated PSFPs based on historical user data (e.g., automatically define a geofence according to time and/or activate data, automatically define associated PSFPs based on historical user selections). Accordingly, various embodiments may advantageously provide user customized experiences with reduced or eliminated user intervention required. Various embodiments may advantageously provide increased user implementation by automatically providing SPEMFs with reduced or eliminated user memory and/or (directed) action requirements.

In some implementations, a mobile app that controls an SFTD may include a subscription model. For example, the subscription model may allow users to access periodic updates of PSFPs. In some implementations, the SFTD may include a security system. For example, the SFTD may require a user to be authenticated before connecting with a portable device of the user. For example, the security system may advantageously promote safety and privacy for the user. The security system may, for example, operate (e.g., lock, unlock) in response to biometric data.

Various embodiments may be provided with features or design elements to promote user convenience or comfort. For example, in some embodiments, a coil may be provided with a soft-touch housing to enhance user comfort when positioning the coil on or about the body. Some embodiments may be provided in a wearable format which, for example, may include straps, bands, and/or slings.

Some embodiments may be voice activated. For example, the SFTD, an application running on a portable device, and/or a separate remote control may be responsive to (predetermined) voice commands

In various embodiments, the SFTDs may be connected to other computing devices. For example, a user may connect the SFTDs wirelessly with a personal computer. For example, a user may connect the SFTDs wirelessly with a smart-TV. For example, a user may connect the SFTDs wirelessly with a smart speaker (e.g., to generate audio signals corresponding to a selected PSFP).

Various embodiments may be provided with additional environmental enhancement components or features to supplement the symphonic PEMF generated according to selected PSFPs. For example, the SFTD may be provided with and/or connected to ambiance lighting circuitry. The SFTD may, for example, be operably coupled to a speaker(s) to play audible music. Some PSFPs may contain associated commands to control lighting and play background music. Some SFTDs may be provided with a controller configured to generate audible and visual feedback directly correlated with a current PSFP (e.g., by scaling the output signal into a visible frequency range, an audible frequency range, or both). Some systems may contain apparatus for virtual reality (VR) for advanced relaxation and enjoyment.

Some embodiments, for example, may be configured to generate music simultaneously with a currently activated PSFP. The music may, for example, be generated according to (predetermined) user preferences. The music may, for example, be automatically selected and/or generated according to a (predetermined) desired effect. For example, music associated with calming (e.g., by historical biofeedback data) may be selected when a CALM PSFP is active.

In various embodiments, a computing device (e.g. laptop, computer, smart home hub, virtual assistant device) may be directed by an app containing at least one program of instructions to direct the portable device to send PSFPs to an SFTD. The computing device may, for example, be implemented as a portable computing device (e.g., smartphone, tablet, smart watch). The program of instructions (e.g., mobile app) may be configured to receive user input while browsing, searching, exploring, and/or otherwise locating and selecting a ‘library’ or ‘store’ of PSFPs. The app may be configured to receive input from the user selecting one of more PSFPs and/or criteria for searching PSFPs. The app may be configured to generate a request message(s) to a remote storage. The app may be further configured to receive a response message and download one or more PSFPs. Some combination of the app, SFTD, and remote storage may be configured to communicate at least some PSFPs via encrypted messages which may, for example, be advantageous in preserving proprietary PSFPs. In some embodiments, PSFPs may be provided to users in encrypted data stores (e.g., removable storage, or storage embedded in the SFTD and decryptable only by firmware installed on the SFTD).

In various embodiments, PSFPs may be provided as one or more frequency sets. PSFPs may be provided in a proprietary nomenclature which may, for example, be only decipherable by the SFTD. The proprietary nomenclature may, for example, define one or more frequency sets, each frequency set selecting one or more frequencies available for the SFTD to generate. The nomenclature may, for example, also predetermine coefficients sufficient to determine the relative amplitude of each frequency or frequency set. In some embodiments, a command from a user's personal device may, for example, simply select (e.g., by a proprietary unique identifying nomenclature) a predetermined frequency set or group of frequency sets preloaded on the SFTD firmware. In some embodiments, a command from a user's personal device may, for example, select one or more predetermined frequency sets or groups of frequency sets and provide relative scaling coefficients (e.g., amplitude multipliers) therefor.

In various embodiments, PSFPs may be designed to define associated SPEMFs which may be advantageously employed for specific situations, for example, to achieve specific therapeutic purposes. PSFPs may be designed for specific therapeutic purposes. Illustrative purposes include, but are not limited to, post-exercise therapy, deep sleep encouragement, bone fracture healing, headache, mental stimulation, post-surgery healing, dental health, heart health, malignant body reduction, reduction or reversal of aging effects, Lyme disease therapy, and normalization of cell growth. Specific PSFPs may be designed and selected to encourage increased or properly balanced adenosine triphosphate (ATP) activity in at least one group of cells. Some PSFPs may be designed specifically for human therapy, veterinary therapy, plant therapy, biological therapy, environmental therapy related to living objects or products thereof (e.g., mold reduction or elimination, and food preparation), or some combination thereof. In some embodiments, specific PSFPs may be designed to activate collagen receptors and/or moisture receptors in a user's face. Such facially directed PSFPs may, for example be advantageously used as a regenerative and/or optimization tool to advantageously allow users to perform a non-invasive “facelift.”

Illustrative specific purposes for individual or classes of PSFPs include, but are not limited to, sports performance optimization and peak performance, energy enhancement, beauty enhancement (e.g., cell rejuvenation, anti-aging, hydration, energy face lift, treatment of blemishes, age spots, and acne), physical longevity, increased vitality, general and/or specific organ or system wellness, specific condition and/or disease therapy (e.g., Lyme disease, cancer, pandemic diseases such as COVID-19), or some combination thereof. Further illustrative uses of specific PSPFs include improving plant production (e.g., infusing green houses with beneficial symphonic PEMFs, for example, to increase plant vitality or to increase production), promotion of animal health or production (which may be used in place of artificial hormone therapies and other unnatural and unhealthy methods), or some combination thereof.

Some PSFPs may be provided free of charge. Some PSFPs may be bundled with another purchase. Some PSFPs may be available for purchase individually and/or in bundles. PSFPs may be organized by category, tagged with multiple associations according to therapy purpose, environment, subject type (e.g., human, adult, child, athlete, pet, or plant), or some combination thereof. Some PSFPs may be tuned to a user. For example, PSFPs may be tuned by a practitioner. PSFPs may, for example, be tuned by an automatic process receiving user-specific input, such as voice, heartbeat, or personal electromagnetic field. The automatic process may, for example, generate from the user-specific input at least one custom PSFP and/or may generate a custom adaptation of at least one existing PSFP.

In some embodiments a PSFP may include an array of data points defining parameters such as frequency, amplitude, and phase of the various sine waves which should make up the SPEMF at discrete points in time. A SFTD may be configured, for example, to jump from one to the next and/or to interpolate therebetween. In some embodiments a PSFP may include function(s) defining continuously varying parameters. In some embodiments, a plurality of frequency sets may be physically embodied in a SFTD. A PSFP may contain commands for combining various sets or parts therefore with scaling factors varying in time for each set.

In various embodiments a SPEMF therapy device may advantageously generate complex formulas configured to achieve a desired benefit(s). For example, 724 frequencies in a specific combination may be required for a cell to produce one molecule of amino acid that attracts a white blood cell to the repair process. Different targets (e.g., users) may respond differently to various frequency combinations. For example, in a range of frequencies associated with a specific effect (e.g., initiating a physiological repair process), one user may respond most strongly to a subset of the frequencies, and another user may respond most strongly to a different subset of frequencies.

Accordingly, various PSFPs may define a combination of frequencies configured to evoke a specific outcome and/or combination of outcomes. The frequency set may, for example, include subsets of frequencies in specific ‘layers’ (e.g., time orders). For example, of at least one 50 frequencies, different frequencies may be present at different time points. In some embodiments at least 50 frequencies may be present simultaneously. In some embodiments, at least 5 frequencies may be present simultaneously. At least 10 frequencies may, for example, may be present simultaneously. At least 20 frequencies may, for example, may be present simultaneously. At least 40 frequencies may, for example, may be present simultaneously. At least 75 frequencies may, for example, may be present simultaneously. Accordingly, various embodiments may advantageously achieve desired effects requiring a complex combination of frequencies. Various embodiments may advantageously provide a range of frequencies such that different users, responding to different subsets of frequencies, may advantageously receive a like desired effect(s).

Various embodiments may advantageously compress a required time of exposure to a SPEMF. For example, at least fifty frequencies delivered simultaneously or semi-simultaneously may reduce a necessary time of delivery from individual delivery of the frequencies. A specific user may, for example, be time-sensitive to receiving frequencies (e.g., sensitivity may decrease as time increases). For example, extended running times may cause ‘overload’ or ‘over saturation’ of a user's body. Accordingly, various embodiments may advantageously compress the running time of a SPEMF. Various embodiments may advantageously provide a desired number of frequencies (e.g., sufficient frequencies to achieve a desired result requiring a large number of frequencies) while compressing the running time sufficiently short to avoid overload and/or over saturation of the body.

Each individual target (e.g., user) may vibrate in health and/or disease at a specific frequency (or combination of frequencies). Accordingly, providing an increased range of frequencies simultaneously may advantageously allow a body/energy field to receive an optimal combination of frequencies. The optimal combination of frequencies may advantageously enable the body to heal quickly based on a specific, custom frequency (sub)set which may be different from those required by another “individual”. Accordingly, various embodiments may increase efficacy of a SPEMF for a desired effect across a broader range of targets (e.g., users).

Multiple frequencies may, for example, be synergistic. For example, a first frequency or group of frequencies may provide a first specific effect. A second frequency or group of frequencies may provide a second specific effect. When delivered simultaneously, the first and second frequencies or groups of frequencies may provide the first effect and the second effect. The first effect and the second effect may be synergistic (e.g., increased response, decreased side effects). In some embodiments the first effect and second effect, when simultaneously induced, may, for example, induce a third effect. Accordingly, various embodiments may advantageously provide synergistic effects in, for example, a single therapy session by providing a symphonic PEMF according to one or more PSFPs.

In various embodiments, a PSFP may include ‘pointer’ frequencies and/or frequency sequences configured to instruct a body where a frequency set is to be received and/or what systems it is to affect. Accordingly, in various embodiments a SPEMF may, for example, provide targeted effects and/or increased efficacy by simultaneously providing specific pointer frequencies in combination with other (predetermined) frequencies configured to induce desired effects.

Various embodiments (e.g., such as disclosed at least with reference to FIG. 6) may be configured to achieve a desired magnetic field (e.g., a time-varying SPEMF defined by a corresponding PSFP). The coil may, for example, be modelled as a substantially inductive load having a reactance that scales proportionally to frequency. A control and/or driver (e.g., SFTD) may be defined by a transfer function “T”. Each frequency component may, for example, be scaled inversely to T (e.g., 1/T) to compensate for frequency-dependent inductance in order to achieve a desired magnetic field. At each frequency in the symphonic set, the SFTD may be configured to scale an output voltage to compensate for the reactance of the inductive load at each frequency component.

Coefficients for a symphonic set may, for example, be based on a predetermined relationship (e.g., corresponding to a PSFP). In some embodiments, a normalization module may be configured to normalize each of the frequency components of a frequency set (e.g., defined by a PSFP). The normalization module may advantageously normalize the amplitude and/or phase of the output magnetic field (e.g., SPEMF) as a function of a frequency corresponding to each component. For example, the normalization module may generate a normalization drive voltage relative to a nominal frequency.

In some examples, the normalization module may generate at least one coefficient to scale an amplitude of each component such that the magnetic field output at that frequency has a predetermined (e.g., 1 per unit or 1 p.u.), or “normalized,” amplitude. The normalized amplitude may be based, for example, on the output magnetic field amplitude (e.g., magnetic field intensity) at a predetermined nominal frequency (e.g., 1 kHz); in such an example, the magnetic field amplitude at the nominal frequency with a nominal (e.g., 1.0 p.u.) command signal (e.g., voltage) may be defined as a 1.0 p.u. amplitude of the magnetic field strength. Similarly, in some examples, the normalization module may generate a coefficient to shift a phase angle of each component such that the magnetic field output at that frequency has a predetermined (e.g., 1 per unit or 1 p.u.), or “normalized,” phase relative to the phase (e.g., a 0-degree phase point) of the nominal magnetic field waveform. Accordingly, coefficients (e.g., defined by a PSFP) may be applied to each frequency component to generate relative amplitudes and/or phases. Accordingly, various embodiments may advantageously produce a SPEMF with a controlled amplitude and/or phase at each frequency as defined by a PSFP.

Some aspects of embodiments may be implemented as a computer system. For example, various implementations may include digital and/or analog circuitry, computer hardware, firmware, software, or combinations thereof. Apparatus elements can be implemented in a computer program product tangibly embodied in an information carrier, e.g., in a machine-readable storage device, for execution by a programmable processor; and methods can be performed by a programmable processor executing a program of instructions to perform functions of various embodiments by operating on input data and generating an output. Some embodiments may be implemented advantageously in one or more computer programs that are executable on a programmable system including at least one programmable processor coupled to receive data and instructions from, and to transmit data and instructions to, a data storage system, at least one input device, and/or at least one output device. A computer program is a set of instructions that can be used, directly or indirectly, in a computer to perform a certain activity or bring about a certain result. A computer program can be written in any form of programming language, including compiled or interpreted languages, and it can be deployed in any form, including as a stand-alone program or as a module, component, subroutine, or other unit suitable for use in a computing environment.

Suitable processors for the execution of a program of instructions include, by way of example and not limitation, both general and special purpose microprocessors, which may include a single processor or one of multiple processors of any kind of computer. Generally, a processor will receive instructions and data from a read-only memory or a random-access memory or both. The essential elements of a computer are a processor for executing instructions and one or more memories for storing instructions and data. Storage devices suitable for tangibly embodying computer program instructions and data include all forms of non-volatile memory, including, by way of example, semiconductor memory devices, such as EPROM, EEPROM, and flash memory devices; magnetic disks, such as internal hard disks and removable disks; magneto-optical disks; and, CD-ROM and DVD-ROM disks. The processor and the memory can be supplemented by, or incorporated in, ASICs (application-specific integrated circuits). In some embodiments, the processor and the memory can be supplemented by, or incorporated in hardware programmable devices, such as FPGAs, for example.

As described herein, the data store may generally include one or more data storage devices that tangibly contain at least a portion of the executable instructions that may be executed on one or more processors to perform the operations described herein.

In some implementations, each system may be programmed with the same or similar information and/or initialized with substantially identical information stored in volatile and/or non-volatile memory. For example, one data interface may be configured to perform auto configuration, auto download, and/or auto update functions when coupled to an appropriate host device, such as a desktop computer or a server.

In some implementations, one or more user-interface features may be custom configured to perform specific functions. An exemplary embodiment may be implemented in a computer system that includes a graphical user interface and/or an Internet browser. To provide for interaction with a user, some implementations may be implemented on a computer having a display device, such as an LCD (liquid crystal display) monitor for displaying information to the user, a keyboard, and a pointing device, such as a mouse or a trackball by which the user can provide input to the computer.

In various implementations, the system may communicate using suitable communication methods, equipment, and techniques. For example, the system may communicate with compatible devices (e.g., devices capable of transferring data to and/or from the system) using point-to-point communication in which a message is transported directly from a source to a receiver over a dedicated physical link (e.g., fiber optic link, infrared link, ultrasonic link, point-to-point wiring, daisy-chain). The components of the system may exchange information by any form or medium of analog or digital data communication, including packet-based messages on a communication network. Examples of communication networks include, e.g., a LAN (local area network), a WAN (wide area network), MAN (metropolitan area network), wireless and/or optical networks, and the computers and networks forming the Internet. Other implementations may transport messages by broadcasting to all or substantially all devices that are coupled together by a communication network, for example, by using omni-directional radio frequency (RF) signals. Still other implementations may transport messages characterized by high directivity, such as RF signals transmitted using directional (i.e., narrow beam) antennas or infrared signals that may optionally be used with focusing optics. Still other implementations are possible using appropriate interfaces and protocols such as, by way of example and not intended to be limiting, USB 2.0, FireWire, ATA/IDE, RS-232, RS-422, RS-485, 802.11 a/b/g/n, Wi-Fi, WiFi-Direct, Li-Fi, BlueTooth, Ethernet, IrDA, FDDI (fiber distributed data interface), token-ring networks, or multiplexing techniques based on frequency, time, or code division. Some implementations may optionally incorporate features such as error checking and correction (ECC) for data integrity, or security measures, such as encryption (e.g., WEP) and password protection.

In various embodiments, a computer system may include non-transitory memory. The memory may be connected to the one or more processors, which may be configured for storing data and computer readable instructions, including processor executable program instructions. The data and computer readable instructions may be accessible to the one or more processors. The processor executable program instructions, when executed by the one or more processors, may cause the one or more processors to perform various operations.

In various embodiments, the computer system may include Internet of Things (IoT) devices. IoT devices may include objects embedded with electronics, software, sensors, actuators, and network connectivity which enable these objects to collect and exchange data. IoT devices may be in-use with wired or wireless devices by sending data through an interface to another device. IoT devices may collect useful data and then autonomously flow the data between other devices.

A number of implementations have been described. Nevertheless, it will be understood that various modifications may be made. For example, advantageous results may be achieved if the steps of the disclosed techniques were performed in a different sequence, or if components of the disclosed systems were combined in a different manner, or if the components were supplemented with other components. In some examples, various steps of operations may be optional or not required. In various implementations, various components or process steps may be combined or integrated with one another. Accordingly, other implementations are contemplated within the scope of the following claims.

What is claimed is:

1. A system for generating symphonic pulsed electromagnetic fields (PEMFs), the system comprising:
a handheld solenoidal coil;
a data store comprising a library of predetermined symphonic frequency profiles (PSFPs) defining a plurality of time varying symphonic PEMFs; and
a control unit configured to drive the handheld solenoidal coil to generate a symphonic PEMF; wherein, in response to an activation signal, the control unit performs symphonic PEMF generation operations, the operations comprising:
receive, in response to a selection signal, at least one PSFP from the library; and
drive the handheld solenoidal coil to generate a time-varying symphonic PEMF defined by the at least one PSFP, the time-varying symphonic PEMF comprising at least 50 frequencies, the at least 50 frequencies comprising frequencies selected from each of four decades starting at 10 Hz.

2. The system of claim 1, wherein:
the selection signal is generated by a portable device in wireless communication between the control unit and the data store,
the portable device is provided with a program of instructions tangibly embodied on a computer readable medium, and,
when the instructions are executed on a processor, the portable device causes control operations to be performed to generate the symphonic PEMF, the control operations comprising:
select the at least one PSFP from the data store;
retrieve the at least one PSFP from the data store;
transmit the at least one PSFP to the control unit; and,
activate the at least one PSFP on the control unit such that the control unit drives the handheld solenoidal coil to generate the time-varying symphonic PEMF defined by the at least one PSFP.

3. The system of claim 2, wherein, the control operations further comprise user-interface operations, the user-interface operations comprising:
display a plurality of PSFPs;
receive a user input corresponding to a user selected PSFP; and
perform the control operations based on the user selected PSFP as the at least one PSFP.

4. The system of claim 2, wherein:
the portable device is further in wireless communication with a recommendation module,
the control operations further include PSFP recommendation operations, the PSFP recommendation operations comprising:
provide metadata to the recommendation module; and
receive a signal corresponding to at least one recommended PSFP from the recommendation module based on the metadata.

5. The system of claim 4, wherein the metadata includes biofeedback data.

6. The system of claim 5, wherein the portable device is operably coupled to at least one sensor configured to capture the biofeedback data.

7. The system of claim 4, wherein the metadata includes behavioral information associated with a user of the system.

8. The system of claim 4, wherein the portable device is configured to generate a display of the at least one recommended PSFP for user selection.

9. The system of claim 1, wherein the at least one PSFP includes frequency, phase, and amplitude parameters for multiple sine waves corresponding to the at least 50 frequencies.

10. A system for generating symphonic pulsed electromagnetic fields (PEMFs), the system comprising:
a solenoidal coil;
a data store comprising a library of predetermined symphonic frequency profiles (PSFPs) defining a plurality of time varying symphonic PEMFs; and
a control unit configured to drive the solenoidal coil to generate a symphonic PEMF, wherein, in response to an activation signal, the control unit performs symphonic PEMF generation operations, the operations comprising:
receive, in response to a selection signal, at least one PSFP from the library; and,
drive the solenoidal coil to generate a time-varying symphonic PEMF defined by the at least one PSFP, the time-varying symphonic PEMF comprising at least 50 frequencies, the at least 50 frequencies comprising frequencies selected from each of four decades starting at 10 Hz.

11. The system of claim 10, wherein the solenoidal coil is configured to be held in a human hand.

12. The system of claim 10, wherein, the control operations further comprise user-interface operations, the user-interface operations comprising:
display a plurality of PSFPs;
receive a user input corresponding to a user selected PSFP; and
perform the control operations based on the user selected PSFP as the at least one PSFP.

13. The system of claim 10, wherein:
the selection signal is generated by a portable device in wireless communication between the control unit and the data store,
the portable device is provided with a program of instructions tangibly embodied on a computer readable medium, and,
when the instructions are executed on a processor, the portable device causes control operations to be performed to generate the symphonic PEMF, the control operations comprising:
select the at least one PSFP from the data store;
retrieve the at least one PSFP from the data store;
transmit the at least one PSFP to the control unit; and,
activate the at least one PSFP on the control unit such that the control unit drives the solenoidal coil to generate the time-varying symphonic PEMF defined by the at least one PSFP.

14. The system of claim 13, wherein:
the portable device is further in wireless communication with a recommendation module,
the control operations further include PSFP recommendation operations, the PSFP recommendation operations comprising:
provide metadata to the recommendation module; and
receive a signal corresponding to at least one recommended PSFP from the recommendation module based on the metadata.

15. The system of claim 14, wherein the metadata includes biofeedback data.

16. The system of claim 15, wherein the portable device is operably coupled to at least one sensor configured to capture the biofeedback data.

17. The system of claim 14, wherein the metadata includes behavioral information associated with a user of the system.

18. The system of claim 14, wherein the portable device is configured to generate a display of the at least one recommended PSFP for user selection.

19. The system of claim 10, wherein the control unit further comprising a status indicator to indicate a status of the control unit, wherein the status comprises:
downloading a PSFP;
ready to deliver a downloaded PSFP; and
currently driving the downloaded PSFP.

20. The system of claim 10, wherein the at least one PSFP includes frequency, phase, and amplitude parameters for multiple sine waves corresponding to the at least 50 frequencies.
